(19) United States
(12) Patent Application Publication
(10) Pub. No.: US 2007/0253954 A1
(43) Pub. Date: Nov. 1, 2007

(54) Epha4 As Therapeutic Target Of Prc And Pdaca

(75) Inventors: Yusuke Nakamura, Yokohama-shi (JP); Hidewaki Nakagawa, Shinagawa-ku (JP); Shuichi Nakatsuru, Saitama-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ONCOTHERAPY SCIENCE, INC., KAWASAKI-SHI (JP)

(21) Appl. No.: 10/590,801

(22) PCT Filed: Feb. 18, 2005
(86) PCT No.: PCT/JP05/03081

§ 371 Date: Jul. 9, 2007

Related U.S. Application Data
(60) Provisional application No. 60/548,335, filed on Feb. 27, 2004.
(60) Provisional application No. 60/555,809, filed on Mar. 24, 2004.

(51) Int. Cl. A61K 31/7088; A61K 39/00; A61K 39/395; A61P 35/00; C07H 21/02; C07H 21/04; C12N 15/63; C12Q 1/48; C12Q 1/68; G01N 33/00

(52) U.S. Cl. 424/138.1; 424/277.1; 435/15; 435/6; 436/501; 436/86; 436/94

(57) ABSTRACT

Objective methods for diagnosing a predisposition to developing prostate cancer (PRC) are described herein. In one embodiment, the diagnostic method involves the determining a expression level of EphA4. The present invention further provides methods of screening for therapeutic agents useful in the treatment of PRC, methods of treating PRC. The invention also features a method for inhibiting growth of a cancer cell by contacting the cell with a composition of a siRNA of EPHA4. Methods of treating cancer are also within the invention. The invention also features products, including nucleic acid sequences and vectors as well as to compositions comprising them, useful in the provided methods. The invention also provides a method for inhibiting of tumor cell, for example pancreatic cancer cell, particularly pancreatic ductal adenocarcinoma (PDACa).

This application claims the benefit of U.S. Provisional Application Ser. No. 60/548,335 filed Feb. 27, 2004 and U.S. Provisional Application Ser. No. 60/555,809 filed Mar. 24, 2004, the contents of which are hereby incorporated by reference in its entirety.

### TECHNICAL FIELD

The present invention relates to methods of detecting and diagnosing a predisposition to developing prostate cancer (PRC) and pancreatic ductal adenocarcinoma (PDACa). The present invention also relates to methods of treating and preventing prostate cancer and pancreatic ductal adenocarcinoma (PDACa). In particular, the present invention relates to EphA4.

### BACKGROUND ART

Prostate cancer (PRC) is one of the most common malignancy in males and the second-leading cause of cancer-related deaths in the United States and Europe (Gronberg et al., 2003). The testing for prostate specific antigen (PSA) in serum can detect early stage of PRC and it is now a gold standard to screen PRC in the high-risk population.

Incidence of prostate cancer is increasing steadily in developed countries according to the prevalence of Western-style diet and increasing number of senior population. Early diagnosis through serum testing for prostate specific antigen (PSA) provides an opportunity for curative surgery and has significantly improved the prognosis of prostate cancer, but up to 30% of patients treated with radical prostatectomy relapse their cancer (Han et al., 2001). Most relapsed or advanced cancers respond to androgen ablation therapy because prostate cancer growth is initially androgen-dependent. However, they eventually progress to androgen-independent disease, at which point they are no longer responsive to androgen ablation therapy. The most serious clinical problem of prostate cancer is that androgen-independent prostate cancer is unresponsive to any other therapies (Gronberg, 2003), and establishing new therapies other than androgen ablation therapy against prostate cancer are urgent issue for management of prostate cancer.

High-grade prostatic intraepithelial neoplasia (PIN) is widely accepted as the main premalignant lesion without invasion of the basal membrane of the acini, which has the potential to progress to invasive PRC (McNeal and Bostwick et al. 1986, DeMarzo et al. 2003, Abate-Shen et al. 2000, Montironi et al. 2002). PIN does not significantly elevate serum PSA concentration and cannot be detected by ultrasound.

High-grade PIN has a high predictive value as a marker for PRC, and its identification warrants repeat biopsy for concurrent or subsequent invasive PRC. Only prostate needle biopsy can recognize this minimal lesions and its identification warrants repeat biopsy for concurrent or subsequent invasive PRC (Bostwick 2000). Performing saturation prostate biopsies to rule out any coexistent prostate cancer followed by every 3-6 month serial repeated prostate biopsies is currently the only way in which to manage patients found to have high-grade PIN. But the reliability of this diagnosis is highly dependent on the technique of prostate needle biopsy, histological processing, and experience of reviewing pathologists (van der Kwast et al. 2003). They cannot perfectly discriminate PRC lesions from PRC nor identify the patients with invasive PRC among the high-risk people with PINs.

Hence accurate identification of PINs and PRC and understanding the prostatic carcinogenesis through PINs are important to avoid error in the diagnosis of invasive PRC and in patient management (Steiner 2001). However, the natural history of PINs and molecular mechanism of the putative transition form PINs to PRC remains unclear and it is still controversial whether these PIN lesions without PRC should be treated or not.

Alternatively, pancreatic ductal adenocarcinoma (PDACa) is the fifth leading cause of cancer death in the western world and has one of the highest mortality rates of any malignancy, with only a 5-year survival rate. In the USA, each year, an estimated 30,700 patients are diagnosed with pancreatic cancer and nearly 30,000 will die of these diseases. The vast majority of patients are diagnosed at an advanced stage of disease, which is unresponsive to current therapies and the patients can survive for a few months. Only surgical resection can offer the possibility of cure, but only 10-20% of patients with PDACa can undergo potentially curative resection and even after curative surgery, 80-90% of the patients relapse and die of the disease. Some improvements in surgical outcome or quality of life occur in patients who also receive chemotherapy including gemcitabine and/or radiation, although the impact on long-term survival has been minimal due to the intense resistance of PDACa to any treatment. At this point, management of most patients focuses on palliation.

Therefore, establishment of a novel molecular therapy for PDACa and identification of novel therapeutic molecular targets for PDACa are urgent issues for pancreatic cancer treatment now.

cDNA microarray technologies have enabled comprehensive profiles of gene expression in normal and malignant cells, and comparison of the gene expression in malignant and corresponding normal cells (Okabe et al., Cancer Res 61:2129-37 (2001); Kitahara et al., Cancer Res 61: 3544-9 (2001); Lin et al., Oncogene 21:4120-8 (2002); Hasegawa et al., Cancer Res 62:7012-7 (2002)). This approach enables an understanding of the complex nature of cancer cells, and helps to understand the mechanism of carcinogenesis. Identification of genes that are deregulated in tumors can lead to more precise and accurate diagnosis of individual cancers, and to develop novel therapeutic targets (Bienz and Clevers, Cell 103:311-20 (2000)). To disclose mechanisms underlying tumors from a genome-wide point of view, and discover target molecules for diagnosis and development of novel therapeutic drugs, the present inventors have been analyzing the expression profiles of tumor cells using a cDNA microarray of 23040 genes (Okabe et al., Cancer Res 61:2129-37 (2001); Kitahara et al., Cancer Res 61:3544-9 (2001); Lin et al., Oncogene 21:4120-8 (2002); Hasegawa et al., Cancer Res 62:7012-7 (2002)).

Studies designed to reveal mechanisms of carcinogenesis have already facilitated identification of molecular targets for anti-tumor agents. For example, inhibitors of farnesyltransferase (FTIs) which were originally developed to inhibit the growth-signaling pathway related to Ras, whose activation depends on posttranslational farnesylation, has been effective in treating Ras-dependent tumors in animal models (He et al., Cell 99:335-45 (1999)). Clinical trials on human using a combination of anti-cancer drugs and anti-HER2 monoclonal antibody, trastuzumab, have been conducted to antagonize the proto-oncogene receptor HER2/neu; and have been achieving improved clinical response and overall survival of breast-cancer patients (Lin et al., Cancer Res 61:6345-9 (2001)). A tyrosine kinase inhibitor, STI-571, which selectively inactivates bcr-abl fusion proteins, has been developed to treat chronic myelogenous leukemias wherein constitutive activation of bcr-abl tyrosine kinase plays a crucial role in the transformation of leukocytes. Agents of these kinds are designed to suppress oncogenic activity of specific gene products (Fujita et al., Cancer Res 61:7722-6 (2001)). Therefore, gene products commonly up-regulated in cancerous cells may serve as potential targets for developing novel anti-cancer agents.

It has been demonstrated that CD8+ cytotoxic T lymphocytes (CTLs) recognize epitope peptides derived from tumor-associated antigens (TAAs) presented on MHC Class I molecule, and lyse tumor cells. Since the discovery of MAGE family as the first example of TAAs, many other TAAs have been discovered using immunological approaches (Boon, Int J Cancer 54: 177-80 (1993); Boon and van der Bruggen, J Exp Med 183: 725-9 (1996); van der Bruggen et al., Science 254: 1643-7 (1991); Brichard et al., J Exp Med 178:489-95 (1993); Kawakami et al., J Exp Med 180: 347-52 (1994)). Some of the discovered TAAs are now in the stage of clinical development as targets of immunotherapy. TAAs discovered so far include MAGE (van der Bruggen et al., Science 254: 1643-7 (1991)), gp100 (Kawakami et al., J Exp Med 180: 347-52 (1994)), SART (Shichijo et al., J Exp Med 187: 277-88 (1998)), and NY-ESO-1 (Chen et al., Proc Natl Acad Sci USA 94: 1914-8 (1997)). On the other hand, gene products which had been demonstrated to be specifically over-expressed in tumor cells, have been shown to be recognized as targets inducing cellular immune responses. Such gene products include p53 (Umano et al., Brit J Cancer 84: 1052-7 (2001)), HER2/neu (Tanaka et al., Brit J Cancer 84: 94-9 (2001)), CEA (Nukaya et al., Int J Cancer 80: 92-7 (1999)), and so on.

In spite of significant progress in basic and clinical research concerning TAAs (Rosenberg et al., Nature Med 3: 321-7 (1998); Mukherji et al., Proc Natl Acad Sci USA 92: 8078-82 (1995); Hu et al., Cancer Res 56: 2479-83 (1996)), only limited number of candidate TAAs for the treatment of adenocarcinomas are available. TAAs abundantly expressed in cancer cells, and at the same time which expression is restricted to cancer cells would be promising candidates as immunotherapeutic targets. Further, identification of new TAAs inducing potent and specific antitumor immune responses is expected to encourage clinical use of peptide vaccination strategy in various types of cancer (Boon and can der Bruggen, J Exp Med 183: 725-9 (1996); van der Bruggen et al., Science 254: 1643-7 (1991); Brichard et al., J Exp Med 178:489-95 (1993); Kawakami et al., J Exp Med 180: 347-52 (1994); Shichijo et al., J Exp Med 187: 277-88 (1998); Chen et al., Proc Natl Acad Sci USA 94: 1914-8 (1997); Harris, J Natl Cancer Inst 88: 1442-5 (1996); Butterfield et al., Cancer Res 59: 3134-42 (1999); Vissers et al., Cancer Res 59: 5554-9 (1999); van der Burg et al., J Immunol 156: 3308-14 (1996); Tanaka et al., Cancer Res 57:4465-8 (1997); Fujie et al., Int J Cancer 80: 169-72 (1999); Kikuchi et al., Int J Cancer 81:459-466 (1999); Oiso et al., Int J Cancer 81: 387-94 (1999)).

It has been repeatedly reported that peptide-stimulated peripheral blood mononuclear cells (PBMCs) from certain healthy donors produce significant levels of IFN-γ in response to the peptide, but rarely exert cytotoxicity against tumor cells in an HLA-A24 or -A0201 restricted manner in ⁵¹Cr-release assays (Kawano et al., Cancer Res 60: 3550-8 (2000); Nishizaka et al., Cancer Res 60:4830-7 (2000); Tamura et al., Jpn J Cancer Res 92: 762-7 (2001)). However, both of HLA-A24 and HLA-A0201 are one of the most common HLA alleles in Japanese, as well as Caucasian populations (Date et al., Tissue Antigens 47: 93-101 (1996); Kondo et al., J Immunol 155:4307-12 (1995); Kubo et al., J Immunol 152: 3913-24 (1994); Imanishi et al., Proceeding of the eleventh International Hictocompatibility Workshop and Conference Oxford University Press, Oxford, 1065 (1992); Williams et al., Tissue Antigens 49: 129-33 (1997)). Thus, antigenic peptides of carcinomas presented by these HLAs may be especially useful for the treatment of carcinomas among Japanese and Caucasian populations. Further, it is known that the induction of low-affinity CTL in vitro usually results from the use of peptide at a high concentration, generating a high level of specific peptide/MHC complexes on antigen presenting cells (APCs), which will effectively activate these CTL (Alexander-Miller et al., Proc Natl Acad Sci USA 93:4102-7 (1996)).

### SUMMARY OF THE INVENTION

The invention is based in part on the discovery that the gene encoding EphA4 is over-expressed in prostate cancer or pancreatic ductal adenocarcinoma (PDACa) compared to non-cancerous tissue. The cDNA of EphA4 is 3468 nucleotides in length. The nucleic acid and polypeptide sequences of EphA4 are shown in SEQ ID NO: 1 and 2, respectively. The sequence data are also available via following accession numbers.

EphA4: L36645, NM_{—}004438

Accordingly, the invention features a method of diagnosing or determining a predisposition to PRC in a subject by determining an expression level of EphA4 in a patient derived biological sample, such as tissue sample. An alteration, e.g., increase of the level of expression of EphA4 compared to a normal control level indicates that the subject suffers from or is at risk of developing PRC.

In the context of the present invention, the phrase “control level” refers to a protein or gene expression level detected in a control sample and includes a normal control level. A control level can be a single expression pattern derived from a single reference population or from a plurality of expression patterns. For example, the control level can be a database of expression patterns from previously tested cells. A “normal control level” refers to a level of gene or protein expression detected in a normal, healthy individual or in a population of individuals known not to be suffering from PRC. A normal individual is one with no clinical symptoms of PRC and PIN.

An increase in the expression level of EphA4 detected in a test sample as compared to a normal control level indicates that the subject (from which the sample was obtained) suffers from or is at risk of developing PRC.

According to the present invention, gene expression level is deemed “altered” when gene expression is increased or decreased 10%, 25%, 50% or more as compared to a normal control level. Alternately, the gene expression may be also be deemed to be altered if gene expression is increased or decreased 1, 2, 5 or more fold as compared to a normal control level. Expression is determined by detecting selective hybridization, e.g., on an array, of EphA4 probe to a gene transcript of the patient-derived tissue sample.

In the context of the present invention, the patient derived tissue sample is any tissue obtained from a test subject, e.g., a patient known to or suspected of having PRC. For example, the tissue may contain an epithelial cell. More particularly, the tissue may be an epithelial cell from prostate tissue.

The present invention further provides methods of identifying an agent that inhibits or enhances the expression of EphA4 gene or the activity of its gene product by contacting a test cell expressing EphA4 gene with a test agent and determining the expression level of EphA4 gene or the activity of its gene product. The test cell may be an epithelial cell, such as an epithelial cell obtained from prostate and pancreatic tissue. A decrease in the expression level of EphA4 gene or biological activity its gene product as compared to that of EphA4 gene or gene product in PRC indicates that the test agent is an inhibitor of expression or function of the EphA4 gene and may be used to reduce a symptom of PRC.

In the present invention, EphA4 can preferably be used as an up regulated marker gene. Moreover, a decrease of the expression level or biological activity in the presence of the agent compared to that in the absence of the test agent indicates the agent is an inhibitor of EphA4 gene and useful to inhibit PRC.

The present invention also provides a kit comprising a detection reagent which binds to EphA4 polynucleotides or EphA4 polypeptides.

The EphA4 gene can also provide information to identify novel chemo-preventive drugs for PRC transformation, and these chemo-preventive drugs can be administered to the selected high-risk population of PRC, that is, those with high-grade PINs, for the purpose of treating or preventing PRC.

Therapeutic methods of the present invention include a method of treating or preventing PRC in a subject including the step of by administering to the subject an inhibitory nucleic acid (e.g., an antisense siRNA, or ribozyme) composition. In the context of the present invention, the antisense composition reduces the expression of the specific target gene. For example, the antisense composition may contain a nucleotide, which is complementary to EphA4 gene sequence. Alternatively, the present method may include the steps of administering to a subject a small interfering RNA (siRNA) composition. In the context of the present invention, the siRNA composition reduces the expression of EphA4 nucleic acid. In yet another method, the treatment or prevention of PRC in a subject may be carried out by administering to a subject a ribozyme composition. In the context of the present invention, the nucleic acid-specific ribozyme composition reduces the expression of EphA4 nucleic acid.

The present invention provides methods for inhibiting cell growth. Among the methods provided are those comprising contacting a cell with a composition comprising a small interfering RNA (siRNA) of EphA4. The invention also provides methods for inhibiting tumor cell growth in a subject. Such methods include administering to a subject a composition comprising a small interfering RNA (siRNA) of EphA4. Another aspect of the invention provides methods for inhibiting the expression of the EphA4 gene in a cell of a biological sample. Expression of the gene may be inhibited by introduction of a double stranded ribonucleic acid (RNA) molecule into the cell in an amount sufficient to inhibit expression of the EphA4 gene. Another aspect of the invention relates to products including nucleic acid sequences and vectors as well as to compositions comprising them, useful, for example, in the provided methods. Among the products provided are siRNA molecules having the property to inhibit expression of the EphA4 gene when introduced into a cell expressing said gene. Among such molecules are those that comprise a sense strand and an antisense strand, wherein the sense strand comprises a ribonucleotide sequence corresponding to a EphA4 target sequence, and wherein the antisense strand comprises a ribonucleotide sequence which is complementary to said sense strand. The sense and the antisense strands of the molecule hybridize to each other to form a double-stranded molecule.

The present invention also includes vaccines and vaccination methods. For example, a method of treating or preventing PRC in a subject may involve administering to the subject a vaccine containing a polypeptide encoded by a nucleic acid of EphA4 or an immunologically active fragment such a polypeptide. In some embodiments, a nucleic acid molecule encoding an EphA4 polypeptide or fragment thereof is administered to the patient. In the context of the present invention, an immunologically active fragment is a polypeptide that is shorter in length than the full-length naturally-occurring protein and yet which induces an immune response analogous to that induced by the full-length protein. For example, an immunologically active fragment should be at least 8 residues in length and capable of stimulating an immune cell such as a T cell or a B cell. Immune cell stimulation can be measured by detecting cell proliferation, elaboration of cytokines (e.g., IL-2), or production of an antibody.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference herein in their entirety. In case of conflict, the present specification, including definitions, will control. In addition, the materials, methods, and examples are illustrative only and not intended to be limiting.

One advantage of the methods described herein is that the disease is identified prior to detection of overt clinical symptoms. Other features and advantages of the invention will be apparent from the following detailed description, and from the claims.

### BRIEF DESCRIPTION OF THE FIGURES

FIG. 1A are photographs showing the results of immunohistochemical analysis of gene that were identified to be differentially expressed in the transition from PIN to PRC. The EphA4 protein was also strongly expressed in PRC cells, while PINs and normal prostatic epithelium (N) from the same patient showed no or very weak expression of EphA4 protein. The PRC, PIN and normal prostate epithelium were included on one prostate cancer tissue. Magnification, ×200.

FIG. 1B are photographs showing the result of immunohistochemistry in PDACa tissues. Over-expression of EphA4 protein was observed in pancreatic ductal adenocarcinoma, but not in normal pancreatic duct.

FIG. 2 depicts photographs of Northern blot analysis showing EphA4 expression pattern in normal adult tissue samples. EphA4 is abundant only in adult testis, suggesting that targeting for EphA4 would be expected to lead less toxicity in human body.

FIG. 3 depicts photographs showing the effect of Knocking-down endogenous EphA4 in prostate cancer cell line, PC3, and in PDACa cell, MIA-Paca2, by siRNA.

FIG. 3 (A) shows the results of RT-PCR. It validated knockdown effect of EphA4 mRNA by transfection of siRNA expression vectors 1313si, but not by EGFPsi. 1313si were designed specifically for EphA4 mRNA sequence, and EGFPsi was for EGFP mRNA sequence. RNA was harvested 8 hours after transfection and analyzed. β2-MG and ACTB were used to normalize input cDNA.

FIG. 3 (B) is a photograph showing the results of Colony formation assay. It showed drastic decrease of colony numbers in the cells one week after transfection with 1313si that were validated to knock down EphA4 effectively by RT-PCR.

FIG. 3 (C) is a photograph showing the results MTT assay. It also showed drastic decreased number of the grown cells transfected with 1313si, but not with EGFPsi.

### DISCLOSURE OF THE INVENTION

The words “a,”, “an” and “the” as used herein mean “at least one” unless otherwise specifically indicated.

As used herein, the term “organism” refers to any living entity comprised of at least one cell. A living organism can be as simple as, for example, a single eukaryotic cell or as complex as a mammal, including a human being.

As used herein, the term “biological sample” refers to a whole organism or a subset of its tissues, cells or component parts (e.g. bodily fluids, including but not limited to blood, mucus, lymphatic fluid, synovial fluid, cerebrospinal fluid, saliva, amniotic fluid, amniotic cord blood, urine, vaginal fluid and semen). “Biological sample” further refers to a homogenate, lysate, extract, cell culture or tissue culture prepared from a whole organism or a subset of its cells, tissues or component parts, or a fraction or portion thereof. Lastly, “biological sample” refers to a medium, such as a nutrient broth or gel in which an organism has been propagated, which contains cellular components, such as proteins or nucleic acid molecules.

The invention is based in part on the discovery that the gene encoding EphA4 is over-expressed in pancreatic ductal adenocarcinoma (PDACa) and prostate cancer (PRC) compared to non-cancerous tissue. The cDNA of EphA4 is 3468 nucleotides in length. The nucleic acid and polypeptide sequences of EphA4 are shown in SEQ ID NO: 1 and 2, respectively. The sequence data are also available via following accession numbers.

EphA4: L36645, NM_{—}004438

EphA4 is one of the family of receptors with tyrosine kinase activity. Their function with their ephrin ligands is well studied in the nervous system, where Eph receptors and ephrin molecules are involved in patterning the developing hindbrain, axon pathfinding and guiding neural crest cell migration (Dodelet V C, and Pasquale E B. Eph receptors and ephrin lignads: embryogenesis to tumorigenesis. Oncogene, 19: 5614-5619, 2000). These molecules also regulate embryonic vascular development and there are some reports about the association of Eph/ephrin with tumor angiogenesis (Dodelet V C, and Pasquale E B. Eph receptors and ephrin lignads: embryogenesis to tumorigenesis. Oncogene, 19: 5614-5619, 2000). The Eph receptor family consists of 13 members and their ligands, ephrins, are divided into two subclasses, the A-subclass (A1-A5) and the B-subclass (B1-B3). The receptors are divided on the basis of sequence similarity and ligand affinity into A-subclass (EphA41-A8), and B-subclass (EphB1-B4, B6). A-type receptors typically bind to most or all A-type ligands, and B-type receptors bind to most or all B-type ligands, with the exception of EphA4 that can bind both A-type and most B-type ligands (Dodelet V C, and Pasquale E B. Eph receptors and ephrin lignads: embryogenesis to tumorigenesis. Oncogene, 19: 5614-5619, 2000).

The differentially expressed genes identified herein are used for diagnostic purposes as markers of predisposition to developing PRC and as gene targets, the expression of which is altered to treat or alleviate a symptom of PRC. The term “predisposition” as used herein indicates a potential to develop PRC.

By measuring expression of the EphA4 gene in a sample of cells, PRC ban be diagnosed. Similarly, measuring the expression of EphA4 gene in response to various agents can identify agents for treating PRC.

The present invention involves determining (e.g., measuring) the expression of EphA4. Using sequence information provided by the GeneBank™ database entries for known sequences, EphA4 gene can be detected and measured using techniques well known to one of ordinary skill in the art. For example, sequences within the sequence database entries corresponding to EphA4 gene, can be used to construct probes for detecting RNA sequences corresponding to EphA4 gene in, e.g., Northern blot hybridization analyses. Probes typically include at least 10, at least 20, at least 50, at least 100, at least 200 nucleotides of a reference sequence. As another example, the sequences can be used to construct primers for specifically amplifying the EphA4 nucleic acid in, e.g., amplification-based detection methods such as reverse-transcription based polymerase chain reaction.

The probes used to detect EphA4 mRNA sequences are typically designed to selectively hybridize to the target mRNA. The term “selective hybridization” and related terms refer to the ability of probe and its target to hybridize under stringent conditions. For example, hybridization may be performed by conducting prehybridization at 68° C. for 30 min or longer using “Rapid-hyb buffer” (Amersham LIFE SCIENCE), adding a labeled probe, and warming at 68° C. for 1 hour or longer. The following washing step can be conducted, for example, in a low stringent condition. A low stringent condition is, for example, 42° C., 2×SSC, 0.1% SDS, or preferably 50° C., 2×SSC, 0.1% SDS. More preferably, high stringent conditions are used. A high stringent condition is, for example, washing 3 times in 2×SSC, 0.01% SDS at room temperature for 20 min, then washing 3 times in 1×SSC, 0.1% SDS at 37° C. for 20 min, and washing twice in 1×SSC, 0.1% SDS at 50° C. for 20 min. However, several factors, such as temperature and salt concentration, can influence the stringency of hybridization and one skilled in the art can suitably select the factors to achieve the requisite stringency.

Expression levels of EphA4 gene in a test cell population, e.g., a patient-derived tissues sample, are then compared to the expression level of the same gene in a reference population. The reference cell population includes one or more cells for which the compared parameter is known. The expression level of EphA4 gene in the specimens from the test cell population and reference cell population may be determined at the same time. Alternatively, expression levels of EphA4 gene in reference cell population can be determined by a statistical method based on the results obtained by analyzing the expression level of the gene in specimens previously collected prostate ductal carcinoma cells (e.g., PRC cells) or normal prostate ductal epithelial cells (e.g., non-PRC cells).

Whether or not a level of gene expression in a test cell population as compared to a reference cell population indicates a predisposition to developing PRC. When the expression level of the gene in a test cell population does not fall within the range of reference cell population, the subject is judged to have high risk to develop PRC.

Moreover, if the reference cell population is made up of PRC cells, a similarity in gene expression profile between the test cell population and the reference cell population indicates that the test cell population includes PRC cells.

A level of expression of EphA4 gene in a test cell population is considered “altered” if it varies from the expression level of the corresponding EphA4 gene in a reference cell population by more than 1.1, more than 1.5, more than 2.0, more than 5.0, more than 10.0 or more fold.

Differential gene expression between a test cell population and a reference cell population can be normalized to a control nucleic acid, e.g. a housekeeping gene. For example, a control nucleic acid is one which is known not to differ depending on the cancerous or non-cancerous state of the cell. The expression level of a control nucleic acid in the test and reference cell population can be used to normalize signal levels in the test and reference populations. Exemplary control genes include, but are not limited to, e.g., β-actin, glyceraldehyde 3-phosphate dehydrogenase and ribosomal protein P1.

The test cell population can be compared to multiple reference cell populations. Each of the multiple reference populations may differ in the known parameter. Thus, a test cell population may be compared to a first reference cell population known to contain, e.g., PRC cells, as well as a second reference population known to contain, e.g., non-PRC cells (normal cells). The test cell may be included in a tissue type or cell sample from a subject known to contain, or suspected of containing, PRC cells.

The test cell is obtained from a bodily tissue or a bodily fluid, e.g., biological fluid (such as blood or sputum, for example). For example, the test cell may be purified from prostate tissue. Preferably, the test cell population comprises an epithelial cell. The epithelial cell is preferably from a tissue known to be or suspected to be cancerous. Cells in the reference cell population should be derived from a tissue type similar that of the test cell. Optionally, the reference cell population is a cell line, e.g. a PRC cell line (i.e., a positive control) or a normal non-PRC cell line (i.e., a negative control). Alternatively, the control cell population may be derived from a database of molecular information derived from cells for which the assayed parameter or condition is known.

The subject is preferably a mammal. Exemplary mammals include, but are not limited to, e.g., a human, non-human primate, mouse, rat, dog, cat, horse, or cow.

Expression of the gene disclosed herein can be determined at the protein or nucleic acid level using methods known in the art. For example, Northern hybridization analysis using probes which specifically recognize (i.e., selectively hybridize to) the nucleic acid sequence of this invention can be used to determine gene expression. Alternatively, gene expression may be measured using reverse-transcription-based PCR assays, e.g., using primers specific for the EphA4 gene sequence. Expression may also be determined at the protein level, i.e., by measuring the level of a polypeptides encoded by EphA4 gene, or biological activity thereof. Such methods are well known in the art and include, but are not limited to, e.g., immunoassays that utilize antibodies to proteins encoded by the genes. The biological activities of the proteins encoded by the genes are generally well known.

Diagnosing PRC

In the context of the present invention, PRC is diagnosed by measuring the expression level of EphA4 polynucleotides from a test population of cells, (i.e., a patient-derived biological sample). Preferably, the test cell population contains an epithelial cell, e.g., a cell obtained from prostate tissue. Gene expression can also be measured from blood or other bodily fluids such as urine. Other biological samples can be used for measuring protein levels. For example, the protein level in blood or serum derived from a subject to be diagnosed can be measured by immunoassay or other conventional biological assay.

Expression of EphA4 gene is determined in the test cell or biological sample and compared to the normal control expression level associated with the EphA4 gene assayed. A normal control level is an expression profile of EphA4 gene typically found in a population known not to be suffering from PRC. An alteration (e.g., an increase or a decrease) in the level of expression in the patient-derived tissue sample of EphA4 gene indicates that the subject is suffering from or is at risk of developing PRC. For example, an increase in the expression of EphA4 gene in the test population as compared to the normal control level indicates that the subject is suffering from or is at risk of developing PRC.

Alteration of EphA4 gene in the test population as compared to the normal control level indicates that the subject suffers from or is at risk of developing PRC. For example, alteration of at least 1%, at least 5%, at least 25%, at least 50%, at least 60%, at least 80%, at least 90% or more of EphA4 gene indicates that the subject suffers from or is at risk of developing PRC.

The expression levels of the EphA4 in a particular specimen can be estimated by quantifying mRNA corresponding to or protein encoded by EphA4 gene. Quantification methods for mRNA are known to those skilled in the art. For example, the levels of mRNAs corresponding to EphA4 can be estimated by Northern blotting or RT-PCR. The nucleotide sequence of EphA4 have already been reported. Anyone skilled in the art can design the nucleotide sequences for probes or primers to quantify the EphA4 gene.

Also the expression level of EphA4 can be analyzed based on the activity or quantity of protein encoded by the gene. A method for determining the quantity of the EphA4 protein is shown below. For example, immunoassay method is useful for the determination of the proteins in biological materials. Any biological materials can be used for the determination of the protein or it's activity. For example, blood sample is analyzed for estimation of the protein encoded by a serum marker. On the other hand, a suitable method can be selected for the determination of the activity of a protein encoded by the EphA4.

In the present invention, a diagnostic agent for diagnosing predisposition to developing PRC, is also provided. The diagnostic agent of the present invention comprises a compound that binds to a polynucleotide or a polypeptide of the present invention. Preferably, an oligonucleotide that hybridizes to the polynucleotide of the EphA4, or an antibody that binds to the polypeptide of the EphA4 may be used as such a compound.

Identifying Agents That Inhibit or Enhance EphA4 Gene Expression

An agent that inhibits the expression of EphA4 gene or the activity of its gene product can be identified by contacting a test cell population expressing an EphA4 gene with a test agent and then determining the expression level of the EphA4 gene. A decrease in the level of expression of the EphA4 gene or in the level of activity of its gene product in the presence of the agent as compared to the normal control level (or compared to the expression or activity in the absence of the test agent) indicates that the agent is an inhibitor of EphA4 gene and useful in inhibiting PRC.

The test cell population may be any cell expressing EphA4 gene. For example, the test cell population may contain an epithelial cell, such as a cell derived from prostate tissue. Furthermore, the test cell may be an immortalized cell line derived from a PRC cell. Alternatively, the test cell may be a cell which has been transfected with a EphA4 gene or which has been transfected with a regulatory sequence (e.g. promoter sequence) from a EphA4 gene operably linked to a reporter gene.

Assessing Efficacy of Treatment of PRC in a Subject

The differentially expressed EphA4 gene identified herein also allow for the course of treatment of PRC to be monitored. In this method, a test cell population is provided from a subject undergoing treatment for PRC. If desired, test cell populations are obtained from the subject at various time points, before, during, and/or after treatment. Expression of EphA4 gene, in the cell population, is then determined and compared to a reference cell population which includes cells whose PRC state is known. In the context of the present invention, the reference cells should have not been exposed to the treatment of interest.

If the reference cell population contains no PRC cells, a similarity in the expression of a EphA4 gene in the test cell population and the reference cell population indicates that the treatment of interest is efficacious. However, a difference in the expression of a EphA4 gene in the test population and a normal control cell population indicates a less favorable clinical outcome or prognosis. Similarly, if the reference cell population contains PRC cells, a difference between the expression of a EphA4 gene in the test cell population and the reference cell population indicates that the treatment of interest is efficacious, while a similarity in the expression of a EphA4 gene in the test population and a normal control reference cell population indicates a less favorable clinical outcome or prognosis.

Additionally, the expression level of EphA4 gene determined in a subject-derived biological sample obtained after treatment (i.e., post-treatment levels) can be compared to the expression level of the EphA4 gene determined in a subject-derived biological sample obtained prior to treatment onset (i.e., pre-treatment levels). Since the EphA4 gene is an up-regulated gene, a decrease in the expression level in a post-treatment sample indicates that the treatment of interest is efficacious while an increase or maintenance in the expression level in the post-treatment sample indicates a less favorable clinical outcome or prognosis.

As used herein, the term “efficacious” indicates that the treatment leads to a reduction in the expression of a pathologically up-regulated gene, a decrease in size, prevalence, or metastatic potential of PRC in a subject. When a treatment of interest is applied prophylactically, the term “efficacious” means that the treatment retards or prevents a PRC from forming or retards, prevents, or alleviates a symptom of clinical PRC. Assessment of prostate tumors can be made using standard clinical protocols.

In addition, efficaciousness can be determined in association with any known method for diagnosing or treating PRC. PRC can be diagnosed, for example, by identifying symptomatic anomalies, e.g., weight loss, abdominal pain, back pain, anorexia, nausea, vomiting and generalized malaise, weakness, and jaundice.

Selecting a Therapeutic Agent for Treating PRC That is Appropriate for a Particular Individual

Differences in the genetic makeup of individuals can result in differences in their relative abilities to metabolize various drugs. An agent that is metabolized in a subject to act as an anti-PRC agent can manifest itself by inducing a change in a gene expression pattern in the subject's cells from that characteristic of a cancerous state to a gene expression pattern characteristic of a non-cancerous state. Accordingly, the differentially expressed EphA4 gene disclosed herein allow for a putative therapeutic or prophylactic inhibitor of PRC to be tested in a test cell population from a selected subject in order to determine if the agent is a suitable inhibitor of PRC in the subject.

To identify an inhibitor of PRC, that is appropriate for a specific subject, a test cell population from the subject is exposed to a therapeutic agent, and the expression of EphA4 gene is determined.

In the context of the method of the present invention, the test cell population contains a PRC cell expressing EphA4 gene. Preferably, the test cell is an epithelial cell. For example a test cell population may be incubated in the presence of a candidate agent and the pattern of gene expression of the test sample may be measured and compared to one or more reference profiles, e.g., a PRC reference expression profile or a non-PRC reference expression profile.

A decrease in expression of EphA4 in a test cell population relative to a reference cell population containing PRC indicates that the agent has therapeutic potential.

In the context of the present invention, the test agent can be any compound or composition. Exemplary, the test agents include, but are not limited to, immunomodulatory agents.

Screening Assays for Identifying Therapeutic Agents

The differentially expressed EphA4 gene disclosed herein can also be used to identify candidate therapeutic agents for treating PRC. The method of the present invention involves screening a candidate therapeutic agent to determine if it can convert an expression profile of EphA4 gene characteristic of a PRC state to a gene expression pattern characteristic of a non-PRC state.

In the present invention, EphA4 are useful for screening of therapeutic agent for treating or preventing PRC.

In the instant method, a cell is exposed to a test agent or a plurality of test agents (sequentially or in combination) and the expression of EphA4 in the cell is measured. The expression profile of EphA4 gene assayed in the test population is compared to expression level of the same EphA4 gene in a reference cell population that is not exposed to the test agent.

An agent capable of suppressing the expression of EphA4 gene has potential clinical benefit. Such agents may be further tested for the ability to prevent PRC in animals or test subjects.

In a further embodiment, the present invention provides methods for screening candidate agents which are potential targets in the treatment of PRC. As discussed in detail above, by controlling the expression levels of EphA4 gene or the activities of their gene products, one can control the onset and progression of PRC. Thus, candidate agents, which are potential targets in the treatment of PRC, can be identified through screening methods that use such expression levels and activities of as indices of the cancerous or non-cancerous state. In the context of the present invention, such screening may comprise, for example, the following steps:
a) contacting a test compound with a polypeptide encoded by a polynucleotide of EphA4;
b) detecting the binding activity between the polypeptide and the test compound; and
c) selecting the test compound that binds to the polypeptide.

Alternatively, the screening method of the present invention may comprise the following steps:
a) contacting a candidate compound with a cell expressing EphA4 gene; and
b) selecting the candidate compound that reduces the expression level of EphA4.
   Cells expressing a EphA4 gene include, for example, cell lines established from PRC; such cells can be used for the above screening of the present invention.

Alternatively, the screening method of the present invention may comprise the following steps:
a) contacting a test compound with a polypeptide encoded by a polynucleotide of EphA4;
b) detecting the biological activity of the polypeptide of step (a); and
c) selecting a compound that suppresses the biological activity of the polypeptide encoded by the polynucleotide of EphA4 as compared to the biological activity detected in the absence of the test compound.

A protein for use in the screening method of the present invention can be obtained as a recombinant protein using the nucleotide sequence of the EphA4 gene. Based on the information regarding the EphA4 gene and its encoded protein, one skilled in the art can select any biological activity of the protein as an index for screening and any suitable measurement method to assay for the selected biological activity.

In the present invention, biological activity of EphA4 is preferably tyrosine kinase activity. The skilled artisan can estimate tyrosine kinase activity. For example, contacting a cell expressing EphA4 with test compound at presence of [γ-³²P]-ATP. Then, phosphorylated protein by tyrosine kinase activity of EphA4 are determined. For detection of the phosphorylated protein, SDS-PAGE or immunoprecipitation can be used. Furthermore, an antibody recognizes phosphorylated tyrosine residue can be used for phosphorylated protein level.

Alternatively, the screening method of the present invention may comprise the following steps:
a) contacting a candidate compound with a cell into which a vector comprising the transcriptional regulatory region of EphA4 gene and a reporter gene that is expressed under the control of the transcriptional regulatory region has been introduced
b) measuring the expression or activity of said reporter gene; and
c) selecting the candidate compound that reduces the expression or activity level of said reporter gene as compared to a level in the absence of the test compound.

Suitable reporter genes and host cells are well known in the art. A reporter construct suitable for the screening method of the present invention can be prepared by using the transcriptional regulatory region of a EphA4 gene.

In the method for screening of the present invention, EphA4 can be used as preferable up-regulated marker gene. Furthermore, we here identified a tyrosine kinase receptor, EphA4, as an over-expressed gene specifically in invasive prostate cancer, not in non-invasive precursor PINs (prostatic intraepitherial neoplasia), by using genome-wide cDNA microarray combined with laser microbeam microdissection. The cDNA microarray and immunohistochemistry demonstrated that EphA4 was over-expressed specifically in invasive prostate cancer cells, not in PINs, and Northern blot analysis showed its restricted expression in adult testis. The knocking-down effect by siRNA specific to EphA4 resulted in drastic suppression of prostate cancer cell growth. These findings demonstrate that EphA4 is associated with growth and motility of invasive prostate cancer cells and this tyrosine kinase receptor, EphA4, is conveniently used as a molecular target for novel prostate cancer therapy without drastic side effect. Accordingly, an agent that inhibits tyrosine kinase activity of EphA4 is useful for therapeutic agent for treating or prevention of PRC.

A compound isolated by the screening methods described above serves as a candidate for the development of drugs that inhibit or enhance the activity of the protein encoded by marker gene and can be applied to the treatment or prevention of PRC.

Moreover, compounds in which a part of the structure of the compound inhibiting or enhancing the activity of proteins encoded by marker genes is converted by addition, deletion and/or replacement are also included as the compounds obtainable by the screening method of the present invention.

When administrating a compound isolated by the method of the present invention as a pharmaceutical for humans and other mammals, such as mice, rats, guinea-pigs, rabbits, cats, dogs, sheep, pigs, cattle, monkeys, baboons, and chimpanzees, the isolated compound can be directly administered or can be formulated into a dosage form using known pharmaceutical preparation methods. For example, according to the need, the drugs can be taken orally, as sugar-coated tablets, capsules, elixirs and microcapsules, or non-orally, in the form of injections of sterile solutions or suspensions with water or any other pharmaceutically acceptable liquid. For example, the compounds can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredient contained in such a preparation makes a suitable dosage within the indicated range acquirable.

Examples of additives that can be admixed into tablets and capsules include, but are not limited to, binders, such as gelatin, corn starch, tragacanth gum and arabic gum; excipients, such as crystalline cellulose; swelling agents, such as corn starch, gelatin and alginic acid; lubricants, such as magnesium stearate; sweeteners, such as sucrose, lactose or saccharin; and flavoring agents, such as peppermint, Gaultheria adenothrix oil and cherry. When the unit-dose form is a capsule, a liquid carrier, such as an oil, can be further included in the above ingredients. Sterile composites for injection can be formulated following normal drug implementations using vehicles such as distilled water suitable for injection.

Physiological saline, glucose, and other isotonic liquids including adjuvants, such as D-sorbitol, D-mannose, D-mannitol, and sodium chloride, can be used as aqueous solutions for injection. These can be used in conjunction with suitable solubilizers, such as alcohol, for example ethanol; polyalcohols, such as propylene glycol; and polyethylene glycol; and non-ionic surfactants, such as Polysorbate 80 (TM) and HCO-50.

Sesame oil or soy-bean oil can be used as an oleaginous liquid, may be used in conjunction with benzyl benzoate or benzyl alcohol as a solubilizer and may be formulated with a buffer, such as phosphate buffer and sodium acetate buffer; a pain-killer, such as procaine hydrochloride; a stabilizer, such as benzyl alcohol and phenol; and/or an anti-oxidant. A prepared injection may be filled into a suitable ampoule.

Methods well known to those skilled in the art may be used to administer the pharmaceutical composition of the present invention to patients, for example as an intraarterial, intravenous, or percutaneous injection or as an intranasal, transbronchial, intramuscular or oral administration. The dosage and method of administration vary according to the body-weight and age of a patient and the administration method; however, one skilled in the art can routinely select a suitable method of administration. If said compound is encodable by a DNA, the DNA can be inserted into a vector for gene therapy and the vector administered to a patient to perform the therapy. The dosage and method of administration vary according to the body-weight, age, and symptoms of the patient; however one skilled in the art can suitably select them.

For example, although the dose of a compound that binds to a protein of the present invention and regulates its activity depends on the symptoms, the dose is generally about 0.1 mg to about 100 mg per day, preferably about 1.0 mg to about 50 mg per day and more preferably about 1.0 mg to about 20 mg per day, when administered orally to a normal adult human (weight 60 kg).

When administering the compound parenterally, in the form of an injection to a normal adult human (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. In the case of other animals, the appropriate dosage amount may be routinely calculated by converting to 60 kgs of body-weight.

Assessing the Prognosis of a Subject with PRC

The present invention also provides a method of assessing the prognosis of a subject with PRC including the step of comparing the expression EphA4 gene in a test cell population to the expression of the same EphA4 gene in a reference cell population derived from patients over a spectrum of disease stages. By comparing the gene expression of EphA4 gene in the test cell population and the reference cell population(s), or by comparing the pattern of gene expression over time in test cell populations derived from the subject, the prognosis of the subject can be assessed.

For example, an increase of expression of EphA4 compared to a normal control indicates less favorable prognosis. A decrease in expression of EphA4 indicates a more favorable prognosis for the subject. The classification score (CS) may be use for the comparing the expression profile.

Kits

The present invention also includes a PRC-detection reagent, e.g., a nucleic acid that specifically binds to or identifies EphA4 nucleic acids, such as oligonucleotide sequences which are complementary to a portion of a EphA4 nucleic acid, or an antibody that bind to EphA4 proteins encoded by EphA4 nucleic acid. The detection reagents may be packaged together in the form of a kit. The reagents are packaged in separate containers, e.g., a nucleic acid or antibody (either bound to a solid matrix or packaged separately with reagents for binding them to the matrix), a control reagent (positive and/or negative), and/or a detectable label. Instructions (e.g., written, tape, VCR, CD-ROM, etc.) for carrying out the assay may also be included in the kit. The assay format of the kit may be a Northern hybridization or a sandwich ELISA, both of which are known in the art.

For example, PRC detection reagent may be immobilized on a solid matrix such as a porous strip to form at least one PRC detection site. The measurement or detection region of the porous strip may include a plurality of sites, each containing a nucleic acid. A test strip may also contain sites for negative and/or positive controls. Alternatively, control sites may be located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of PRC present in the sample. The detection sites may be configured in any suitably detectable shape and are typically in the shape of a bar or dot spanning the width of a test strip.

Methods of Inhibiting PRC

The present invention further provides a method for treating or alleviating a symptom of PRC in a subject by decreasing the expression or activity of EphA4 (or the activity of its gene product). Suitable therapeutic compounds can be administered prophylactically or therapeutically to a subject suffering from or at risk of (or susceptible to) developing PRC. Such subjects can be identified using standard clinical methods or by detecting an aberrant level of expression of EphA4 or aberrant activity of its gene product. In the context of the present invention, suitable therapeutic agents include, for example, inhibitors of cell proliferation, and protein kinase activity.

Alternatively, the therapeutic method of the present invention may include the step of decreasing the expression, function, or both, of gene products of gene whose expression is aberrantly increased (“up-regulated” or “over-expressed” gene”) in prostate cells. Expression may be inhibited in any of several ways known in the art. For example, expression can be inhibited by administering to the subject a nucleic acid that inhibits, or antagonizes, the expression of the over-expressed gene, e.g., an antisense oligonucleotide or small interfering RNA which disrupts expression of the over-expressed gene.

Antisense Nucleic Acids:

As noted above, antisense nucleic acids corresponding to the nucleotide sequence of EphA4 can be used to reduce the expression level of EphA4. Antisense nucleic acids corresponding to EphA4 that are up-regulated in PRC are useful for the treatment of PRC. Specifically, the antisense nucleic acids of the present invention may act by binding to EphA4 or mRNAs corresponding thereto, thereby inhibiting the transcription or translation of the gene, promoting the degradation of the mRNAs, and/or inhibiting the expression of proteins encoded by a nucleic acid of EphA4, finally inhibiting the function of the proteins. The term “antisense nucleic acids” as used herein encompasses both nucleotides that are entirely complementary to the target sequence and those having a mismatch of one or more nucleotides, so long as the antisense nucleic acids can specifically hybridize to the target sequences. For example, the antisense nucleic acids of the present invention include polynucleotides that have a homology of at least 70% or higher, preferably at least 80% or higher, more preferably at least 90% or higher, even more preferably at least 95% or higher over a span of at least 15 continuous nucleotides. Algorithms known in the art can be used to determine the homology.

Percent homology (also referred to as percent identity) are typically carried out between two optimally aligned sequences. Methods of alignment of sequences (either polynucleotides or polypeptides) for comparison are well-known in the art. Optimal alignment of sequences and comparison can be conducted, e.g., using the following the algorithm in “Wilbur and Lipman, Proc Natl Acad Sci USA 80: 726-30 (1983)”. As used herein, the terms “substantially identical”, “substantially homologous” and similar terms are used to describe two sequences (polypeptides or polynucleotides) that are at least about 80%, usually about 85%, about 90%, about 95%, about 97%, or about 99% identical using standard sequence comparison algorithms, such as that described above.

The antisense nucleic acid derivatives of the present invention act on cells producing the proteins encoded by EphA4 gene by binding to the DNAs or mRNAs encoding the proteins, inhibiting their transcription or translation, promoting the degradation of the mRNAs, and inhibiting the expression of the proteins, thereby resulting in the inhibition of the protein function.

An antisense nucleic acid derivative of the present invention can be made into an external preparation, such as a liniment or a poultice, by admixing it with a suitable base material which is inactive against the nucleic acid.

Also, as needed, the antisense nucleic acids of the present invention can be formulated into tablets, powders, granules, capsules, liposome capsules, injections, solutions, nose-drops and freeze-drying agents by adding excipients, isotonic agents, solubilizers, stabilizers, preservatives, pain-killers, and such. These can be prepared by following known methods.

The antisense nucleic acids derivative of the present invention can be given to the patient by direct application onto the ailing site or by injection into a blood vessel so that it will reach the site of ailment. An antisense-mounting medium can also be used to increase durability and membrane-permeability. Examples include, but are, not limited to liposomes, poly-L-lysine, lipids, cholesterol, lipofectin or derivatives of these. Furthermore, derivatives or modified products of the antisense-oligonucleotides can also be used in the present invention. Examples of such modified products include lower alkyl phosphonate modifications such as methyl-phosphonate-type or ethyl-phosphonate-type, phosphorothioate modifications and phosphoroamidate modifications.

The dosage of the antisense nucleic acid derivative of the present invention can be adjusted suitably according to the patient's condition and used in desired amounts. For example, a dose range of 0.1 to 100 mg/kg, preferably 0.1 to 50 mg/kg can be administered.

The antisense nucleic acids of the present invention inhibit the expression of a protein of the present invention and are thereby useful for suppressing the biological activity of the protein of the invention. In addition, expression-inhibitors, comprising antisense nucleic acids of the present invention, are useful in that they can inhibit the biological activity of a protein of the present invention.

The method of the present invention can be used to alter the expression in a cell of EphA4 gene. Binding of the antisense nucleic acids to a transcript corresponding to EphA4 in the target cell results in a reduction in the protein production by the cell. The length of the oligonucleotide is at least 10 nucleotides and may be as long as the naturally-occurring transcript. Preferably, the oligonucleotide is about 19 to about 25 nucleotides in length. Most preferably, the oligonucleotide is less than about 75, about 50, or about 25 nucleotides in length.

The antisense nucleic acids of present invention include modified oligonucleotides. For example, thioated oligonucleotides may be used to confer nuclease resistance to an oligonucleotide.

si RNA:

Also, a siRNA against a EphA4 gene can be used to reduce the expression level of the EphA4 gene.

The invention features methods of inhibiting cell growth. Cell growth is inhibited by contacting a cell with a composition of a small interfering RNA (siRNA) of EphA4. The cell is further contacted with a transfection-enhancing agent. The cell is provided in vitro, in vivo or ex vivo. The subject is a mammal, e.g., a human, non-human primate, mouse, rat, dog, cat, horse, or cow. The cell is a pancreatic ductal cell. Alternatively, the cell is a tumor cell (i.e., cancer cell) such as a carcinoma cell or an adenocarcinoma cell. For example, the cell is a pancreatic ductal adenocarcinoma cell. By inhibiting cell growth is meant that the treated cell proliferates at a lower rate or has decreased viability than an untreated cell. Cell growth is measured by proliferation assays known in the art.

Herein, the term “siRNA” refers to a double stranded RNA molecule which prevents translation of a target mRNA. Standard techniques for introducing siRNA into the cell may be used, including those in which DNA is a template from which RNA is transcribed. In the context of the present invention, the siRNA comprises a sense nucleic acid sequence and an anti-sense nucleic acid sequence against an up-regulated gene, such as EphA4. The siRNA is constructed such that a single transcript has both the sense and complementary antisense sequences from the target gene, e.g., a hairpin.

An siRNA of EphA4 hybridizes to target mRNA and thereby decreases or inhibits production of the EphA4 polypeptides encoded by the gene by associating with the normally single-stranded mRNA transcript, thereby interfering with translation and thus, expression of the protein. In the context of the present invention, an siRNA is preferably less than 500, 200, 100, 50, or 25 nucleotides in length. More preferably an siRNA is 19-25 nucleotides in length. In order to enhance the inhibition activity of the siRNA, nucleotide “u” can be added to 3′ end of the antisense strand of the target sequence. The number of “u”s to be added is at least 2, generally 2 to 10, preferably 2 to 5. The added “u”s form single strand at the 3′ end of the antisense strand of the siRNA.

The nucleotide sequence of suitable siRNAs can be designed using an siRNA design computer program available from the Ambion website (http://www.ambion.com/techlib/misc/siRNA_finder.html). The computer program selects nucleotide sequences for siRNA synthesis based on the following protocol.

Selection of siRNA Target Sites:
1. Beginning with the AUG start codon of the object transcript, scan downstream for AA dinucleotide sequences. Record the occurrence of each AA and the 3′ adjacent 19 nucleotides as potential siRNA target sites. Tuschl, et al., don't recommend against designing siRNA to the 5′ and 3′ untranslated regions (UTRs) and regions near the start codon (within 75 bases) as these may be richer in regulatory protein binding sites (Targeted mRNA degradation by double-stranded RNA in vitro. Genes Dev 13(24): 3191-7 (1999)). UTR-binding proteins and/or translation initiation complexes may interfere with binding of the siRNA endonuclease complex.
2. Compare the potential target sites to the human genome database and eliminate from consideration any target sequences with significant homology to other coding sequences. The homology search can be performed using BLAST, which can be found on the NCBI server at: www.ncbi.nlm.nih.gov/BLAST/
3. Select qualifying target sequences for synthesis. At Ambion, preferably several target sequences can be selected along the length of the gene to evaluate.

Also included in the invention are isolated nucleic acid molecules that include the nucleic acid sequence of target sequences, for example, nucleotides of SEQ ID NO: 10 or a nucleic acid molecule that is complementary to the nucleic acid sequence of SEQ ID NO: 10. As used herein, an “isolated nucleic acid” is a nucleic acid removed from its original environment (e.g., the natural environment if naturally occurring) and thus, synthetically altered from its natural state. In the present invention, isolated nucleic acid includes DNA, RNA, and derivatives thereof. When the isolated nucleic acid is RNA or derivatives thereof, base “t” should be replaced with “U” in the nucleotide sequences. As used herein, the term “complementary” refers to Watson-Crick or Hoogsteen base pairing between nucleotides units of a nucleic acid molecule, and the term “binding” means the physical or chemical interaction between two nucleic acids or compounds or associated nucleic acids or compounds or combinations thereof. Complementary nucleic acid sequences hybridize under appropriate conditions to form stable duplexes containing few or no mismatches. Furthermore, the sense strand and antisense strand of the isolated nucleotide of the present invention, can form double stranded nucleotide or hairpin loop structure by the hybridization. In a preferred embodiment, such duplexes contain no more than 1 mismatch for every 10 matches. In an especially preferred embodiment, where the strands of the duplex are fully complementary, such duplexes contain no mismatches. The nucleic acid molecule is less than 3468 nucleotides in length for EphA4. For example, the nucleic acid molecule is less than about 500, about 200, or about 75 nucleotides in length. Also included in the invention is a vector containing one or more of the nucleic acids described herein, and a cell containing the vectors. The isolated nucleic acids of the present invention are useful for siRNA against EphA4, or DNA encoding the siRNA. When the nucleic acids are used for siRNA or coding DNA thereof, the sense strand is preferably longer than about 19 nucleotides, and more preferably longer than about 21 nucleotides.

The antisense oligonucleotide or siRNA of the present invention inhibits the expression of a polypeptide of the present invention and is thereby useful for suppressing the biological activity of a polypeptide of the invention. Also, expression-inhibitors, comprising the antisense oligonucleotide or siRNA of the invention, are useful in the point that they can inhibit the biological activity of the polypeptide of the invention. Therefore, a composition comprising an antisense oligonucleotide or siRNA of the present invention is useful for treating or preventing a PRC.

Methods of Inhibiting Cell Growth:

The present invention relates to inhibiting cell growth, i.e, cancer cell growth by inhibiting expression of EphA4. Expression of EphA4 is inhibited by small interfering RNA (siRNA) that specifically target the EphA4 gene. EphA4 targets include, for example, nucleotides of SEQ ID NO: 10.

In non-mammalian cells, double-stranded RNA (dsRNA) has been shown to exert a strong and specific silencing effect on gene expression, which is referred as RNA interference (RNAi) (Sharp P A. RNAi and double-strand RNA. Genes Dev. 1999 Jan. 15; 13(2):139-41). dsRNA is processed into 20-23 nucleotides dsRNA called small interfering RNA (siRNA) by an enzyme containing RNase III motif. The siRNA specifically targets complementary mRNA with a multicomponent nuclease complex (Hammond S M, Bernstein E, Beach D, Hannon G J. An RNA-directed nuclease mediates post-transcriptional gene silencing in Drosophila cells. Nature. 2000 Mar. 16; 404(6775):293-6., Hannon G J. RNA interference. Nature. 2002 Jul. 11; 418(6894):244-51.). In mammalian cells, siRNA composed of 20 or 21-mer dsRNA with 19 complementary nucleotides and 3′ terminal noncomplementary dimmers of thymidine or uridine, have been shown to have a gene specific knock-down effect without inducing global changes in gene expression (Elbashir S M, Harborth J, Lendeckel W, Yalcin A, Weber K, Tuschl T. Duplexes of 21-nucleotide RNAs mediate RNA interference in cultured mammalian cells. Nature. 2001 May 24; 411(6836):494-8.). In addition, plasmids containing small nuclear RNA (snRNA) U6 or polymerase III H1-RNA promoter effectively produce such short RNA recruiting type III class of RNA polymerase III and thus can constitutively suppress its target mRNA (Miyagishi M, Taira K. U6 promoter-driven siRNAs with four uridine 3′ overhangs efficiently suppress targeted gene expression in mammalian cells. Nat. Biotechnol. 2002 May; 20(5):497-500, Brummelkamp T R, Bernards R, Agami R. A System for Stable Expression of Short Interfering RNAs in Mammalian Cells Science. 296(5567):550-553, Apr. 19, 2002.).

The growth of cells is inhibited by contacting a cell with a composition containing a siRNA of EphA4. The cell is further contacted with a transfection agent. Suitable transfection agents are known in the art. By inhibition of cell growth is meant the cell proliferates at a lower rate or has decreased viability compared to a cell not exposed to the composition. Cell growth is measured by methods known in the art such as, the MTT cell proliferation assay.

The siRNA of EphA4 is directed to a single target of EphA4 gene sequence. Alternatively, the siRNA is directed to multiple target of EphA4 gene sequences. For example, the composition contains siRNA of EphA4 directed to two, three, four, or five or more target sequences of EphA4. By EphA4 target sequence is meant a nucleotide sequence that is identical to a portion of the EphA4 gene. The target sequence can include the 5′ untranslated (UT) region, the open reading frame (ORF) or the 3′ untranslated region of the human EphA4 gene. Alternatively, the siRNA is a nucleic acid sequence complementary to an upstream or downstream modulator of EphA4 gene expression. Examples of upstream and downstream modulators include, a transcription factor that binds the EphA4 gene promoter, a kinase or phosphatase that interacts with the EphA4 polypeptide, a EphA4 promoter or enhancer.

siRNA of EphA4 which selectively hybridizes to target mRNA decrease or inhibit production of the EphA4 polypeptide product encoded by the EphA4 gene by associating with the normally single-stranded mRNA transcript, thereby interfering with translation and thus, expression of the protein. The siRNA is less than about 500, about 200, about 100, about 50, or about 25 nucleotides in length. Preferably the siRNA is 19-25 nucleotides in length. Exemplary nucleic acid sequence for the production of EphA4 siRNA include the sequences of nucleotides of SEQ ID NO: 10 as the target sequence, respectively. Furthermore, in order to enhance the inhibition activity of the siRNA, nucleotide “1” can be added to 3′ end of the antisense strand of the target sequence. The number of “u”s to be added is at least 2, generally 2 to 10, preferably 2 to 5. The added “u”s form single strand at the 3′ end of the antisense strand of the siRNA.

The cell is any cell that expresses or over-expresses EphA4. The cell is an epithelial cell such as a pancreatic ductal cell. Alternatively, the cell is a tumor cell such as a carcinoma, adenocarcinoma, blastoma, leukemia, myeloma, or sarcoma. The cell is a pancreatic ductal adenocarcinoma.

An siRNA of EphA4 is directly introduced into the cells in a form that is capable of binding to the mRNA transcripts. Alternatively, the DNA encoding the siRNA of EphA4 is in a vector.

Vectors are produced for example by cloning a EphA4 target sequence into an expression vector operatively-linked regulatory sequences flanking the EphA4 sequence in a manner that allows for expression (by transcription of the DNA molecule) of both strands (Lee, N. S., Dohjima, T., Bauer, G., Li, H., Li, M.-J., Ehsani, A., Salvaterra, P., and Rossi, J. (2002) Expression of small interfering RNAs targeted against HIV-1 rev transcripts in human cells. Nature Biotechnology 20: 500-505.). An RNA molecule that is antisense to EphA4 mRNA is transcribed by a first promoter (e.g., a promoter sequence 3′ of the cloned DNA) and an RNA molecule that is the sense strand for the EphA4 mRNA is transcribed by a second promoter (e.g., a promoter sequence 5′ of the cloned DNA). The sense and antisense strands hybridize in vivo to generate siRNA constructs for silencing of the EphA4 gene. Alternatively, two constructs are utilized to create the sense and anti-sense strands of a siRNA construct. Cloned EphA4 can encode a construct having secondary structure, e.g., hairpins, wherein a single transcript has both the sense and complementary antisense sequences of the target gene.

A loop sequence consisting of an arbitrary nucleotide sequence can be located between the sense and antisense sequence in order to form the hairpin loop structure. Thus, the present invention also provides siRNA having the general formula 5′-[A]-[B]-[A′]-3′, wherein [A] is a ribonucleotide sequence corresponding to a sequence selected from the group consisting of nucleotides of SEQ ID NO: 10,

[B] is a ribonucleotide sequence consisting of about 3 to about 23 nucleotides, and

[A′] is a ribonucleotide sequence consisting of the complementary sequence of [A].

The region [A] hybridizes to [A′], and then a loop consisting of region [B] is formed. The loop sequence may be preferably 3 to 23 nucleotide in length. The loop sequence, for example, can be selected from group consisting of following sequences (http://www.ambion.com/techlib/tb/tb_{—}506.html). Furthermore, loop sequence consisting of 23 nucleotides also provides active siRNA (Jacque, J.-M., Triques, K., and Stevenson, M. (2002) Modulation of HIV-1 replication by RNA interference. Nature 18:35-438.).

CCC, CCACC or CCACACC: Jacque, J. M., Triques, K., and Stevenson, M (2002) Modulation of HV-1 replication by RNA interference. Nature, Vol. 18:35-438.

UUCG: Lee, N. S., Dohjima, T., Bauer, G., Li, H., Li, M.-J., Ehsani, A., Salvaterra, P., and Rossi, J. (2002) Expression of small interfering RNAs targeted against HIV-1 rev transcripts in human cells. Nature Biotechnology 20: 500-505. Fruscoloni, P., Zamboni, M., and Tocchini-Valentini, G. P. (2003) Exonucleolytic degradation of double-stranded RNA by an activity in Xenopus laevis germinal vesicles. Proc. Natl. Acad. Sci. USA 100(4): 1639-1644.

UUCAAGAGA: Dykxhoorn, D. M., Novina, C. D., and Sharp, P. A. (2002) Killing the messenger: Short RNAs that silence gene expression. Nature Reviews Molecular Cell Biology: 57-467.

For example, preferable siRNAs having hairpin loop structure of the present invention are shown below. In the following structure, the loop sequence can be selected from group consisting of CCC, UUCG, CCACC, CCACACC, and UUCAAGAGA. Preferable loop sequence is UUCAAGAGA (“ttcaagaga” in DNA).

GCAGCACCAUCAUCCAUUG-[B]-CAAUGGAUGAUGGUGCUGC
(for target sequence of SEQ ID NO:10)

The regulatory sequences flanking the EphA4 sequence are identical or are different, such that their expression can be modulated independently, or in a temporal or spatial manner. siRNAs are transcribed intracellularly by cloning the EphA4 gene templates into a vector containing, e.g., a RNA polymerase III transcription unit from the small nuclear RNA (snRNA) U6 or the human H1 RNA promoter. For introducing the vector into the cell, transfection-enhancing agent can be used. FuGENE (Rochediagnostices), Lipofectamine 2000 (Invitrogen), Oligofectamine (Invitrogen), and Nucleofector (Wako pure Chemical) are useful as the transfection-enhancing agent.

Oligonucleotides and oligonucleotides complementary to various portions of EphA4 mRNA were tested in vitro for their ability to decrease production of EphA4 in tumor cells (e.g., using the pancreatic cell line such as pancreatic ductal adenocarcinoma (PDACa) cell line) according to standard methods. A reduction in EphA4 gene product in cells contacted with the candidate siRNA composition compared to cells cultured in the absence of the candidate composition is detected using specific antibodies of EphA4 or other detection strategies. Sequences which decrease production of EphA4 in in vitro cell-based or cell-free assays are then tested for there inhibitory effects on cell growth. Sequences which inhibit cell growth in vitro cell-based assay are test in vivo in rats or mice to conform decreased EphA4 production and decreased tumor cell growth in animals with malignant neoplasms.

Methods of Treating Malignant Tumors:

Patients with tumors characterized as over-expressing EphA4 are treated by administering siRNA of EphA4. siRNA therapy is used to inhibit expression of EphA4 in patients suffering from or at risk of developing, for example, PRC or pancreatic ductal adenocarcinoma (PDACa). Such patients are identified by standard methods of the particular tumor type. PRC or pancreatic ductal adenocarcinoma (PDACa) is diagnosed for example, by CT, MRI, ERCP, MRCP, computer tomography, or ultrasound. Treatment is efficacious if the treatment leads to clinical benefit such as, a reduction in expression of EphA4, or a decrease in size, prevalence, or metastatic potential of the tumor in the subject. When treatment is applied prophylactically, “efficacious” means that the treatment retards or prevents tumors from forming or prevents or alleviates a clinical symptom of the tumor. Efficaciousness is determined in association with any known method for diagnosing or treating the particular tumor type.

siRNA therapy is carried out by administering to a patient a siRNA by standard vectors and/or gene delivery systems, including administration of siRNA molecules that have been modified to prevent degradation in vivo. Suitable gene delivery systems may include liposomes, receptor-mediated delivery systems, or viral vectors such as herpes viruses, retroviruses, adenoviruses and adeno-associated viruses, among others. A therapeutic nucleic acid composition is formulated in a pharmaceutically acceptable carrier. The therapeutic composition may also include a gene delivery system as described above. Pharmaceutically acceptable carriers are biologically compatible vehicles which are suitable for administration to an animal, e.g., physiological saline. A therapeutically effective amount of a compound is an amount which is capable of producing a medically desirable result such as reduced production of a EphA4 gene product, reduction of cell growth, e.g., proliferation, or a reduction in tumor growth in a treated animal.

Parenteral administration, such as intravenous, subcutaneous, intramuscular, and intraperitoneal delivery routes, may be used to deliver siRNA compositions of EphA4. For treatment of pancreatic tumors, direct infusion the celiac artery, splenic artery, or common hepatic artery, is useful.

Dosages for any one patient depends upon many factors, including the patient's size, body surface area, age, the particular nucleic acid to be administered, sex, time and route of administration, general health, and other drugs being administered concurrently. Dosage for intravenous administration of nucleic acids is from approximately 10⁶ to 10²² copies of the nucleic acid molecule.

The polynucleotides are administered by standard methods, such as by injection into the interstitial space of tissues such as muscles or skin, introduction into the circulation or into body cavities or by inhalation or insufflation. Polynucleotides are injected or otherwise delivered to the animal with a pharmaceutically acceptable liquid carrier, e.g., a liquid carrier, which is aqueous or partly aqueous. The polynucleotides are associated with a liposome (e.g., a cationic or anionic liposome). The polynucleotide includes genetic information necessary for expression by a target cell, such as a promoters.

Antibodies:

Alternatively, function of gene products of the gene over-expressed in PRC can be inhibited by administering a compound that binds to or otherwise inhibits the function of the gene products. For example, the compound is an antibody which binds to the over-expressed gene product or gene products. Such a binding agent that specifically recognizes the EphA4 protein could also be, for example, a ligand specific for the protein, or a synthetic polypeptide that specifically binds the protein (see e.g., WO2004044011)

The present invention refers to the use of antibodies, particularly antibodies against a protein encoded by an EphA4, or a fragment of such an antibody. As used herein, the term “antibody” refers to an immunoglobulin molecule having a specific structure, that interacts (i.e., binds) only with the antigen that was used for synthesizing the antibody or with an antigen closely related thereto. Furthermore, an antibody may be a fragment of an antibody or a modified antibody, so long as it binds to the proteins encoded by EphA4 gene. For instance, the antibody fragment may be Fab, F(ab′)₂, Fv, or single chain Fv (scFv), in which Fv fragments from H and L chains are ligated by an appropriate linker (Huston J. S. et al. Proc. Natl. Acad. Sci. U.S.A. 85:5879-5883 (1988)). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co M. S. et al. J. Immunol. 152:2968-2976 (1994); Better M. and Horwitz A. H. Methods Enzymol. 178:476-496 (1989); Pluckthun A. and Skerra A. Methods Enzymol. 178:497-515 (1989); Lamoyi E. Methods Enzymol. 121:652-663 (1986); Rousseaux J. et al. Methods Enzymol. 121:663-669 (1986); Bird R. E. and Walker B. W. Trends Biotechnol. 9:132-137 (1991)).

An antibody may be modified by conjugation with a variety of molecules, such as polyethylene glycol (PEG). The present invention provides such modified antibodies. The modified antibody can be obtained by chemically modifying an antibody. Such modification methods are conventional in the field. Alternatively, an antibody may comprise as a chimeric antibody having a variable region derived from a nonhuman antibody and a constant region derived from a human antibody, or a humanized antibody, comprising a complementarity determining region (CDR) derived from a nonhuman antibody, the frame work region (FR) derived from a human antibody and the constant region. Such antibodies can be prepared by using known technologies. Humanization can be performed by substituting rodent CDRs or CDR sequences for the corresponding sequences of a human antibody (see e.g., Verhoeyen et al., Science 239:1534-1536 (1988)). Accordingly, such humanized antibodies are chimeric antibodies, wherein substantially less than an intact human variable domain has been substituted by the corresponding sequence from a non-human species.

Fully human antibodies comprising human variable regions in addition to human framework and constant regions can also be used. Such antibodies can be produced using various techniques known in the art. For example in vitro methods involve use of recombinant libraries of human antibody fragments displayed on bacteriophage (e.g., Hoogenboom & Winter, J. Mol. Biol. 227:381 (1991), Similarly, human antibodies can be made by introducing of human immunoglobulin loci into transgenic animals, e.g., mice in which the endogenous immunoglobulin genes have been partially or completely inactivated. This approach is described, e.g., in U.S. Pat. Nos. 6,150,584, 5,545,807; 5,545,806; 5,569,825; 5,625,126; 5,633,425; 5,661,016.

Cancer therapies directed at specific molecular alterations that occur in cancer cells have been validated through clinical development and regulatory approval of anti-cancer drugs such as trastuzumab (Herceptin) for the treatment of advanced breast cancer, imatinib methylate (Gleevec) for chronic myeloid leukemia, gefitinib (Iressa) for non-small cell lung cancer (NSCLC), and rituximab (anti-CD20 mAb) for B-cell lymphoma and mantle cell lymphoma (Ciardiello F, Tortora G. A novel approach in the treatment of cancer: targeting the epidermal growth factor receptor. Clin Cancer Res. 2001 October; 7(10):2958-70. Review.; Slamon D J, Leyland-Jones B, Shak S, Fuchs H, Paton V, Bajamonde A, Fleming T, Eiermann W, Wolter J, Pegram M, Baselga J, Norton L. Use of chemotherapy plus a monoclonal antibody against HER2 for metastatic breast cancer that overexpresses HER2. N Engl J. Med. 2001 Mar. 15; 344(11):783-92.; Rehwald U, Schulz H, Reiser M, Sieber M, Staak J O, Morschhauser F, Driessen C, Rudiger T, Muller-Hermelink K, Diehl V, Engert A. Treatment of relapsed CD20+ Hodgkin lymphoma with the monoclonal antibody rituximab is effective and well tolerated: results of a phase 2 trial of the German Hodgkin Lymphoma Study Group. Blood. 2003 Jan. 15; 101(2):420-424.; Fang G, Kim C N, Perkins C L, Ramadevi N, Winton E, Wittmann S and Bhalla K N. (2000). Blood, 96, 2246-2253.). These drugs are clinically effective and better tolerated than traditional anti-cancer agents because they target only transformed cells. Hence, such drugs not only improve survival and quality of life for cancer patients, but also validate the concept of molecularly targeted cancer therapy. Furthermore, targeted drugs can enhance the efficacy of standard chemotherapy when used in combination with it (Gianni L. (2002). Oncology, 63 Suppl 1,7-56.; Klejman A, Rushen L, Morrione A, Slupianek A and Skorski T. (2002). Oncogene, 21, 5868-5876.). Therefore, future cancer treatments will probably involve combining conventional drugs with target-specific agents aimed at different characteristics of tumor cells such as angiogenesis and invasiveness.

These modulatory methods can be performed ex vivo or in vitro (e.g., by culturing the cell with the agent) or, alternatively, in vivo (e.g., by administering the agent to a subject). The methods involve administering a protein or combination of proteins or a nucleic acid molecule or combination of nucleic acid, molecules as therapy to counteract aberrant expression of the differentially expressed genes or aberrant activity of their gene products.

Diseases and disorders that are characterized by increased (relative to a subject not suffering from the disease or disorder) expression levels or biological activity of the gene and gene products, respectively, may be treated with therapeutics that antagonize (i.e., reduce or inhibit) activity of the over-expressed gene or genes. Therapeutics that antagonize activity can be administered therapeutically or prophylactically.

Accordingly, therapeutics that may be utilized in the context of the present invention including, e.g., (i) a polypeptide of the over-expressed or under-expressed gene or genes, or analogs, derivatives, fragments or homologs thereof; (ii) antibodies to the over-expressed gene or gene products; (iii) nucleic acids encoding the under-expressed gene or gene s; (iv) antisense nucleic acids or nucleic acids that are “dysfunctional” (i.e., due to a heterologous insertion within the nucleic acids of one or more over-expressed gene or genes); (v) small interfering RNA (siRNA); or (vi) modulators (i.e., inhibitors, agonists and antagonists that alter the interaction between an over/under-expressed polypeptide and its binding partner). The dysfunctional antisense molecules are utilized to “knockout” endogenous function of a polypeptide by homologous recombination (see, e.g., Capecchi, Science 244: 1288-1292 1989).

Increased level can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a gene whose expression is altered). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression.

Therapeutic methods of the present invention may include the step of contacting a cell with an agent that modulates one or more of the activities of the gene products of the differentially expressed genes. Examples of agent that modulates protein activity include, but are not limited to, a nucleic acids, proteins, a naturally-occurring cognate ligands of such proteins, peptides, a peptidomimetics, and other small molecule. For example, a suitable agent may stimulate one or more protein activities of one or more differentially under-expressed genes.

Vaccinating Against Prostate Cancer:

The present invention also relates to a method of treating or preventing PRC in a subject comprising the step of administering to said subject a vaccine comprising a polypeptide encoded by a nucleic acid of EphA4 or an immunologically active fragment of said polypeptide, or a polynucleotide encoding such a polypeptide or fragment thereof. Vaccines can also be administered as nucleic acid compositions wherein DNA or RNA encoding an EphA4 polypeptides, or a fragment thereof, is administered to a patient. See, e.g., Wolff et. al. (1990) Science 247:1465-1468; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

Polypeptides of the invention can also be expressed by viral or bacterial vectors. Examples of expression vectors include attenuated viral hosts, such as vaccinia or fowlpox. This approach involves the use of vaccinia virus, e.g., as a vector to express nucleotide sequences that encode the EphA4 polypeptides or polypeptide fragments. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover, et al. (1991) Nature 351:456-460. A wide variety of other vectors useful for therapeutic administration or immunization e.g., adeno and adeno-associated virus vectors, retroviral vectors, Salmonella typhi vectors, detoxified anthrax toxin vectors, and the like, will be apparent. See, e.g., Shata, et al. (2000) Mol. Med. Today 6:66-71; Shedlock, et al. (2000) J. Leukoc. Biol. 68:793-806; and Hipp, et al. (2000) In Vivo 14:571-85.

Administration of the polypeptide or nucleic acid induces an anti-tumor immunity in a subject. To induce anti-tumor immunity, a polypeptide encoded by a nucleic acid of EphA4 or an immunologically active fragment of said polypeptide, or a polynucleotide encoding such a or fragment thereof polypeptide is administered to subject in need thereof. The polypeptide or the immunologically active fragments thereof are useful as vaccines against PRC. In some cases, the proteins or fragments thereof may be administered in a form bound to the T cell receptor (TCR) or presented by an antigen presenting cell (APC), such as macrophage, dendritic cell (DC), or B-cells. Due to the strong antigen presenting ability of DC, the use of DC is most preferable among the APCs.

In the present invention, a vaccine against PRC refers to a substance that has the ability to induce anti-tumor immunity upon inoculation into animals. According to the present invention, polypeptides encoded by a nucleic acid of EphA4 or fragments thereof were suggested to be HLA-A24 or HLA-A*0201 restricted epitopes peptides that may induce potent and specific immune response against PRC cells expressing EphA4. Thus, the present invention also encompasses method of inducing anti-tumor immunity using the polypeptides. In general, anti-tumor immunity includes immune responses such as follows:

induction of cytotoxic lymphocytes against tumors,

induction of antibodies that recognize tumors, and

induction of anti-tumor cytokine production.

Therefore, when a certain protein induces any one of these immune responses upon inoculation into an animal, the protein is determined to have anti-tumor immunity inducing effect. The induction of the anti-tumor immunity by a protein can be detected by observing in vivo or in vitro the response of the immune system in the host against the protein.

For example, a method for detecting the induction of cytotoxic T lymphocytes is well known. Specifically a foreign substance that enters the living body is presented to T cells and B cells by the action of antigen presenting cells (APCs). T cells that respond to the antigen presented by the APCs in an antigen specific manner differentiate into cytotoxic T cells (or cytotoxic T lymphocytes; CTLs) due to stimulation by the antigen, and then proliferate (this is referred to as activation of T cells). Therefore, CTL induction by a certain peptide can be evaluated by presenting the peptide to a T cell via an APC, and detecting the induction of CTLs. Furthermore, APCs have the effect of activating CD4+ T cells, CD8+ T cells, macrophages, eosinophils, and NK cells. Since CD4+ T cells and CD8+ T cells are also important in anti-tumor immunity, the anti-tumor immunity inducing action of the peptide can be evaluated using the activation effect of these cells as indicators.

A method for evaluating the inducing action of CTLs using dendritic cells (DCs) as the APC is well known in the art. DCs are a representative APCs having the strongest CTL-inducing action among APCs. In this method, the test polypeptide is initially contacted with DCs, and then the DCs are contacted with T cells. Detection of T cells having cytotoxic effects against the cells of interest after the contact with DC shows that the test polypeptide has an activity of inducing the cytotoxic T cells. Activity of CTLs against tumors can be detected, for example, using the lysis of ⁵¹Cr-labeled tumor cells as the indicator. Alternatively, the method of evaluating the degree of tumor cell damage using ³H-thymidine uptake activity or LDH (lactose dehydrogenase)-release as the indicator is also well known.

Apart from DCs, peripheral blood mononuclear cells (PBMCs) may also be used as the APC. The induction of CTLs has been reported to be enhanced by culturing PBMCs in the presence of GM-CSF and IL-4. Similarly, CTLs have been shown to be induced by culturing PBMCs in the presence of keyhole limpet hemocyanin (KLH) and IL-7.

Test polypeptides confirmed to possess CTL-inducing activity by these methods are deemed to be polypeptides having DC activation effect and subsequent CTL-inducing activity. Therefore, polypeptides that induce CTLs against tumor cells are useful as vaccines against tumors. Furthermore, APCs that have acquired the ability to induce CTLs against tumors through contact with the polypeptides are also useful as vaccines against tumors. Furthermore, CTLs, that have acquired cytotoxicity due to presentation of the polypeptide antigens by APCs can also be used as vaccines against tumors. Such therapeutic methods for tumors using anti-tumor immunity due to APCs and CTLs are referred to as cellular immunotherapy.

Generally, when using a polypeptide for cellular immunotherapy, efficiency of the CTL-induction is known to be increased by combining a plurality of polypeptides having different structures and contacting them with DCs. Therefore, when stimulating DCs with protein fragments, it is advantageous to use a mixture of multiple types of fragments.

Alternatively, the induction of anti-tumor immunity by a polypeptide can be confirmed by observing the induction of antibody production against tumors. For example, when antibodies against a polypeptide are induced in a laboratory animal immunized with the polypeptide, and when growth of tumor cells is suppressed by those antibodies, the polypeptide is deemed to have the ability to induce anti-tumor immunity.

Anti-tumor immunity is induced by administering the vaccine of this invention, and the induction of anti-tumor immunity enables treatment and prevention of PRC. Therapy against cancer or prevention of the onset of cancer includes any of the following steps, such as inhibition of the growth of cancerous cells, involution of cancer, and suppression of occurrence of cancer. A decreases in mortality and mortality of individuals having cancer, decrease in the levels of tumor markers in the blood, alleviation of detectable symptoms accompanying cancer, and such are also included in the therapy or prevention of cancer. Such therapeutic and preventive effects are preferably statistically significant. For example, in observation, at a significance level of 5% or less, wherein the therapeutic or preventive effect of a vaccine against cell proliferative diseases is compared to a control without vaccine administration. For example, Student's t-test, the Mann-Whitney U-test, or ANOVA may be used for statistical analysis.

The above-mentioned protein having immunological activity or a vector encoding the protein may be combined with an adjuvant. An adjuvant refers to a compound that enhances the immune response against the protein when administered together (or successively) with the protein having immunological activity. Exemplary adjuvants include, but are not limited to, cholera toxin, salmonella toxin, alum, and such, but are not limited thereto. Furthermore, the vaccine of this invention may be combined appropriately with a pharmaceutically acceptable carrier. Examples of such carriers includes sterilized water, physiological saline, phosphate buffer, culture fluid, and such. Furthermore, the vaccine may contain as necessary, stabilizers, suspensions, preservatives, surfactants, and such. The vaccine can be administered systemically or locally. Vaccine administration can be performed by single administration, or boosted by multiple administrations.

When using an APC or CTL as the vaccine of this invention, tumors can be treated or prevented, for example, by the ex vivo method. More specifically, PBMCs of the subject receiving treatment or prevention are collected, the cells are contacted with the polypeptide ex vivo, and following the induction of APCs or CTLs, the cells may be administered to the subject. APCs can be also induced by introducing a vector encoding the polypeptide into PBMCs ex vivo. APCs or CTLs induced in vitro can be cloned prior to administration. By cloning and growing cells having high activity of damaging target cells, cellular immunotherapy can be performed more effectively. Furthermore, APCs and CTLs isolated in this manner may be used for cellular immunotherapy not only against individuals from whom the cells are derived, but also against similar types of tumors from other individuals.

Furthermore, a pharmaceutical composition for treating or preventing a cell proliferative disease, such as cancer, comprising a pharmaceutically effective amount of the polypeptide of the present invention is provided. The pharmaceutical composition may be used for raising anti tumor immunity.

Pharmaceutical Compositions for Inhibiting PRC

In the context of the present invention, suitable pharmaceutical formulations include those suitable for oral, rectal, nasal, topical (including buccal and sub-lingual), vaginal or parenteral (including intramuscular, sub-cutaneous and intravenous) administration, or for administration by inhalation or insufflation. Preferably, administration is intravenous. The formulations are optionally packaged in discrete dosage units.

Pharmaceutical formulations suitable for oral administration include capsules, cachets or tablets, each containing a predetermined amount of active ingredient. Suitable formulations also include powders, granules, solutions, suspensions and emulsions. The active ingredient is optionally administered as a bolus electuary or paste. Tablets and capsules for oral administration may contain conventional excipients, such as binding agents, fillers, lubricants, disintegrant and/or wetting agents. A tablet may be made by compression or molding, optionally with one or more formulational ingredients. Compressed tablets may be prepared by compressing in a suitable machine the active ingredients in a free-flowing form, such as a powder or granules, optionally mixed with a binder, lubricant, inert diluent, lubricating, surface active and/or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), and/or preservatives. The tablets may optionally be formulated so as to provide slow or controlled release of the active ingredient therein. A package of tablets may contain one tablet to be taken on each of the month.

Formulations suitable for parenteral administration include aqueous and non-aqueous sterile injection solutions, optionally contain anti-oxidants, buffers, bacteriostats and solutes which render the formulation isotonic with the blood of the intended recipient; as well as aqueous and non-aqueous sterile suspensions including suspending agents and/or thickening agents. The formulations may be presented in unit dose or multi-dose containers, for example as sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations suitable for rectal administration include suppositories with standard carriers such as cocoa butter or polyethylene glycol. Formulations suitable for topical administration in the mouth, for example buccally or sublingually, include lozenges, containing the active ingredient in a flavored base such as sucrose and acacia or tragacanth, and pastilles comprising the active ingredient in a base such as gelatin and glycerin or sucrose and acacia. For intra-nasal administration the compounds of the invention may be used as a liquid spray, a dispersible powder or in the form of drops. Drops may be formulated with an aqueous or non-aqueous base also comprising one or more dispersing agents, solubilizing agents and/or suspending agents.

For administration by inhalation the compounds can be conveniently delivered from an insufflator, nebulizer, pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base such as lactose or starch. The powder composition may be presented in unit dosage form, for example, as capsules, cartridges, gelatin or blister packs from which the powder may be administered with the aid of an inhalator or insufflators.

Other formulations include implantable devices and adhesive patches; which release a therapeutic agent.

When desired, the above described formulations, adapted to give sustained release of the active ingredient, may be employed. The pharmaceutical compositions may also contain other active ingredients such as antimicrobial agents, immunosuppressants and/or preservatives.

It should be understood that in addition to the ingredients particularly mentioned above, the formulations of this invention may include other agents conventional in the art with regard to the type of formulation in question. For example, formulations suitable for oral administration may include flavoring agents.

Preferred unit dosage formulations contain an effective dose, as recited below, or an appropriate fraction thereof, of the active ingredient.

For each of the aforementioned conditions, the compositions, e.g., polypeptides and organic compounds, can be administered orally or via injection at a dose ranging from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The dose employed will depend upon a number of factors, including the age and sex of the subject, the precise disorder being treated, and its severity. Also the route of administration may vary depending upon the condition and its severity. In any event, appropriate and optimum dosages may be routinely calculated by those skilled in the art, taking into consideration the above-mentioned factors.

Aspects of the present invention are described in the following examples, which are not intended to limit the scope of the invention described in the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### BEST MODE FOR CARRYING OUT THE INVENTION

The invention will be further described in the following examples, which do not limit the scope of the invention described in the claims.

### EXAMPLE 1

1. General Methods

Patients and Tissue Samples

Tissue samples were obtained with informed consent from 26 cancer patients undergoing radical prostatectomy. All surgical specimens were at clinical stages T2a-T3a with or without N1, and their Gleason scores were 5-9. Histopathological diagnoses were made by a single pathologist before LMM. All samples were embedded in TissueTek OCT medium (Sakura, Tokyo, Japan) immediately after surgical resection and stored at −80° C. until use. From among the 26 resected tissues, 20 cancers and 10 high-grade PINs had sufficient amounts and quality of RNA for microarray analysis.

Laser Microbeam Microdissection and T7-Based RNA Amplification

LMM and T7-based RNA amplification were performed as described previously. Prostate tumor cells and normal prostatic ductal epithelial cells were isolated selectively using the EZ cut system with a pulsed ultraviolet narrow beam-focus laser (SL Microtest GmbH, Germany) in accordance with the manufacturer's protocols. After DNase treatment, total RNAs were subjected to two rounds of T7-based amplification, which yielded 50-100 μg of aRNA from each sample. Then 2.5 μg aliquots of aRNA from PRC or PIN cells and from normal prostatic ductal epithelial cells were labeled by reverse transcription with Cy5-dCTP (tumor cells) or Cy3-dCTP (normal cells) (Amersham Biosciences, Buckinghamshire, UK), as described previously (Ono et al. 2000).

cDNA Microarray Analysis and Acquisition of Data

We fabricated a genome-wide cDNA microarray with 23,040 cDNAs selected from the UniGene database (build #131) of the National Center for Biotechnology Information (NCBI). Construction, hybridization, washing, and scanning were carried out according to methods described previously (Ono et al. 2000). Signal intensities of Cy3 and Cy5 from the 23,040 spots were quantified and analyzed by substituting backgrounds, using ArrayVision software (Imaging Research, Inc., St. Catharines, Ontario, Canada). Subsequently, the fluorescent intensities of Cy5 (tumor) and Cy3 (control) for each target spot were adjusted so that the mean Cy3/Cy5 ratio of 52 housekeeping genes was equal to one. Because data derived from low signal intensities are less reliable, we determined a cut-off value on each slide (Ono et al. 2000) and excluded genes from further analysis when both Cy3 and Cy5 dyes yielded signal intensities lower than the cut-off. For other genes, we calculated the Cy5/Cy3 ratio using the raw data of each sample.

Identification of Genes That were Up- or Down-Regulated from PINs to PRC

We identified genes with changed expression in 20 PRC and 10 PINs according to the following criteria: 1) genes for which we were able to obtain expression data in more than 50% of the cases examined; and 2) genes whose expression ratio was more than 3.0 in prostate cancers and between 0.5 and 2.0 in PINs (defined as up-regulated genes) or genes whose expression ratio was less than 0.33 in cancers and between 0.5 and 2.0 in PINs (defined as down-regulated genes) in more than 50% of informative cases.

Immunohistochemistry

Formalin-fixed and paraffin-embedded prostatic tumor sections were immunostained using a rabbit anti-EphA4 (EphA4) polyclonal antibody (Santa Cruz Biotechnology Inc., Santa Cruz, Calif.) EphA4 expression. Prostate cancer tissues included PRC cells, PIN cells and normal prostatic epithelium heterogeneously. Deparaffinized tissue sections were placed in 10 mM citrate buffer, pH 6.0, and heated to 108° C. in an autoclave for 15 minutes for antigen retrieval. Sections were incubated with a 1:10 dilution or a 1:100 dilution of primary antibody for EphA4, respectively, in a humidity chamber for an hour at room temperature, and developed with peroxidase labeled-dextran polymer followed by diaminobenzidine (DAKO Envision Plus System; DAKO Corporation, Carpinteria, Calif.). Sections were counterstained with hematoxylin. For negative controls, primary antibody was omitted.

2. Northern-Blot Analysis.

Human multiple-tissue Northern blots (Clontech, Palo Alto, Calif.) were hybridized with a [α-³²P] dCTP-labeled PCR product of EphA4. The 1013-bp PCR products were prepared by RT-PCR using primers:

| | |
|---|---|
| 5′-GAAGGCGTGGTCACTAAATGTAA-3′ | (SEQ ID NO:3) |
| and | |
| 5′-TTTAATTTCAGAGGGCGAAGAC-3′. | (SEQ ID NO:4) |

Pre-hybridization,

hybridization and washing were performed according to the supplier's recommendations. The blots were autoradiographed with intensifying screens at −80° C. for 7 days.

3. siRNA-Expressing Constructs and Colony Formation/MTT Assay.

We used siRNA-expression vector (psiU6BX) for RNAi effect to the target genes. The U6 promoter was cloned into the upstream of the gene specific sequence (19 nt sequence from the target transcript separated by a short spacer TTCAAGAGA (SEQ ID NO:9) from the reverse complement of the same sequence) and five thymidines as a termination signal, furthermore neo cassette was integrated to become resistant to Geneticin (Sigma). The target sequences for EphA4 are

| | |
|---|---|
| 5′-GCAGCACCATCATCCATTG-3′ | (SEQ ID NO:10) (1313si), |
| and | |
| 5′-GAAGCAGCACGACTTCTTC-3′ | (SEQ ID NO:11) (EGFPsi) |

as a negative control. The target sequences were designed against full length sequence of EphA4. The nucleotide sequence of EphA4 and amino acid sequence encoded by the nucleotide sequence were shown as SEQ ID NO:1 and SEQ ID NO:2, respectively (GenBank Accession No. NM_{—}004438). PC3 prostate cancer cell lines were plated onto 10-cm dishes (5×10⁵ cells/dish) and transfected with psiU6BX containing EGFP target sequence (EGFPsi) and psiU6BX containing target sequence using Lipofectamine 2000 (Invitrogen) according to manufacture's instruction. Cells were selected by 500 mg/ml Geneticin for one week, and preliminary cells were harvested 8 hours after transfection and analyzed by RT-PCR to validate knockdown effect on EphA4. The primers of RT-PCR were the same ones described above. These cells were also stained by Giemsa solution and performed MTT assay to evaluate the colony formation and the cell number, respectively.
4. Identification of EphA4 Gene Up-Regulated During Malignant Transformation from PINs to Prostate Cancers

We focused on differential expression patterns between PINs and PRC to search for genes likely to be involved in the transition from non-invasive precursor PINs to malignant cancers. Comparing the expression profiles of 20 PRC with those of 10 PINs, we identified 1 up-regulated gene, EphA4; the altered gene might be involved with cell adhesion or motility in invasive PRC cells. EphA4 is one of the tyrosine kinase receptors and is likely to play a critical role of neuronal circuit development and angiogenesis by regulating cell shape and motility, and its over-expression in PRC is likely to be associated with PRC cell motility (Kullander et al. 2002). Some of the later are associated with cell adhesion and proteinase activity, suggesting that their expression changes may contribute to the invasive phenotype by abolishing ductal structures during the transition from PIN to PRC.

5. Immunohistochemistry

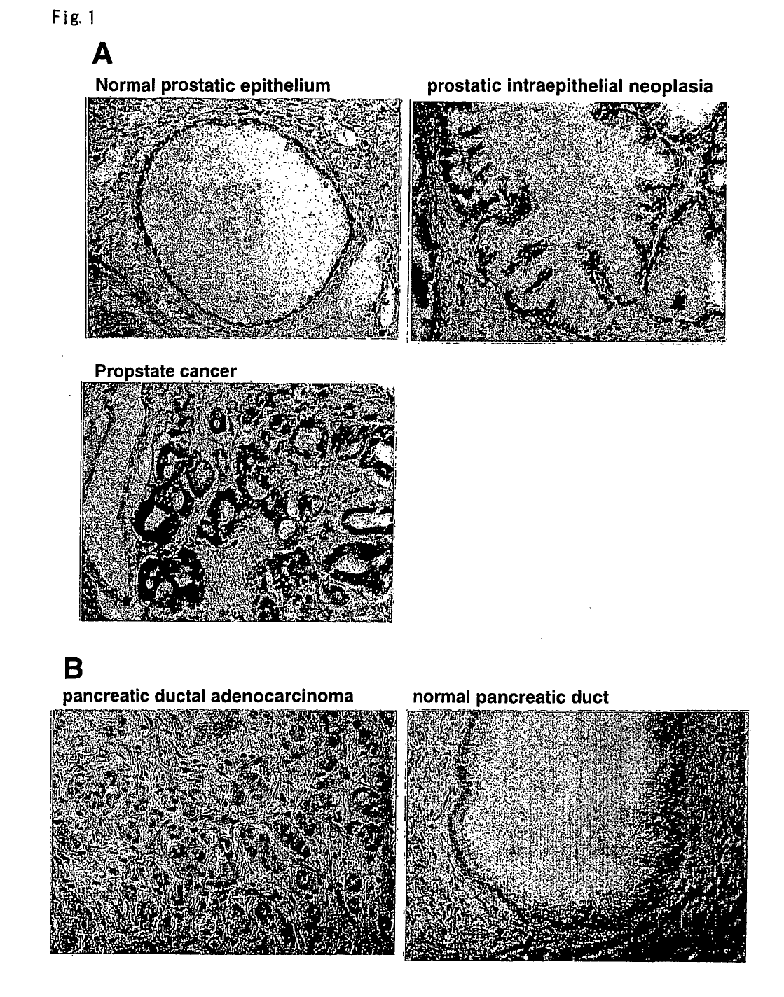

To validate the gene expression pattern in the transition from PIN to PRC, we performed immunohistochemical analysis of the genes differentially expressed in the transition from PIN to PRC in our data. In general, prostate cancer tissues includes PRC cells, PIN cells and normal prostatic epithelium heterogenously, and we compared the staining pattern of each kinds of cells associated with prostatic carcinogenesis on the same tissues from the same patient. As shown in FIG. 1, EphA4 protein was also strongly expressed in PRC cells while PINs and normal prostatic epithelium from the same patient had no or very weak expression of EphA4 protein. The results implicate this expression profile analysis is highly reliable.

We focused on EphA4 because EphA4 is one of the receptors with tyrosine kinase activity and an ideal molecule target for drug design and antibody therapy against cancer. Now a number of tyrosine kinase inhibitors are on clinical trial for cancer treatment, including EGFR (epidermal growth factor receptor) inhibitors, PDGFR (platelet derived growth factor receptor) inhibitors, and VEGF (vascular endothelial growth factor) inhibitors (Dancey and Sausville et al., 2003, Morgan et al., 2003). In addition, trastuzumab (Herceptin), a humanized monoclonal antibody against a tyrosine kinase receptor ERBB2/Her2 (epidermal growth factor receptor 2), is effective for subsets of metastatic breast cancer with HER2 over-expressed (Dancey and Sausville et al., 2003). These tyrosine kinase receptors as drug targets for cancer can be approached by both small molecules and antibody strategy.

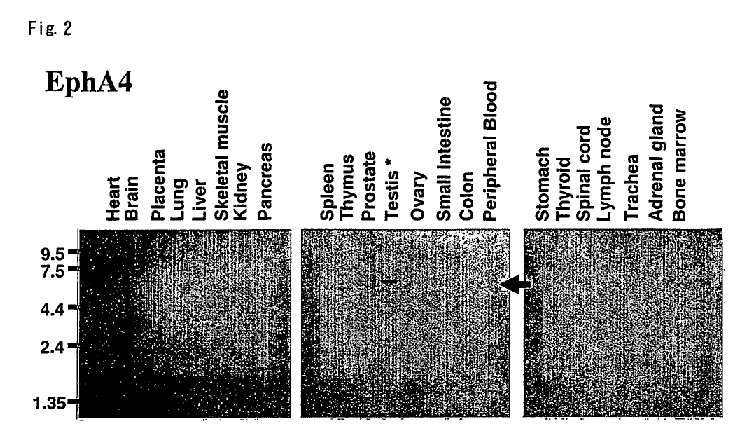

EphA4 is one of the class of receptors with tyrosine kinase activity and their functions with their ephrin ligands are well studied in the nervous system, where Eph receptors and ephrin molecules are involved in patterning the developing hindbrain, axon pathfinding and guiding neural crest cell migration (Dodelet et al., 2000, Kurai and Pasquale, 2003). These molecules also regulate embryonic vascular development and there are some reports about the association of Eph/ephrin with tumor angiogenesis (Gale and Yancopoulos, 1999, Dodelet et al., 2000). The Eph receptor family consists of 13 members and their ligands, ephirins, are divided into two subclasses, the A-subclass (A1-A5) and the B-subclass (B1-B3). The receptors are divided on the basis of sequence similarity and ligand affinity into A-subclass (EphA41-A8), and B-subclass (EphB1-B4, B6). A-type receptors typically bind to most or all A-type ligands, and B-type receptors bind to most or all B-type ligands, with the exception of EphA4 that can bind both A-type and most B-type ligands (Dodelet et al., 2000, Kurai and Pasquale, 2003). In prostate cancer tissues, the ligand of EphA4 is unknown. Northern blot analysis showed that EphA4 was abundant in testis, not in central nervous system and other major organs (FIG. 2). Recently the antibody targeting against other Eph receptor family member, EphA42 that is also over-expressed in several cancers, was reported to inhibit breast cancer cell growth in vitro and in vivo (Carles-Kinch et al., 2002, Coffman et al., 2003). However, EphA42 is expressed ubiquitously in adult tissues, indicating much more possibility of toxicity in treatment of antibody therapy. Considering its tyrosine kinase activity, membrane localization and its restricted expression pattern, EphA4 is one the most ideal molecular targets for prostate cancer.

6. Growth Suppression Mediated by siRNA in Prostate Cancer Cell Lines

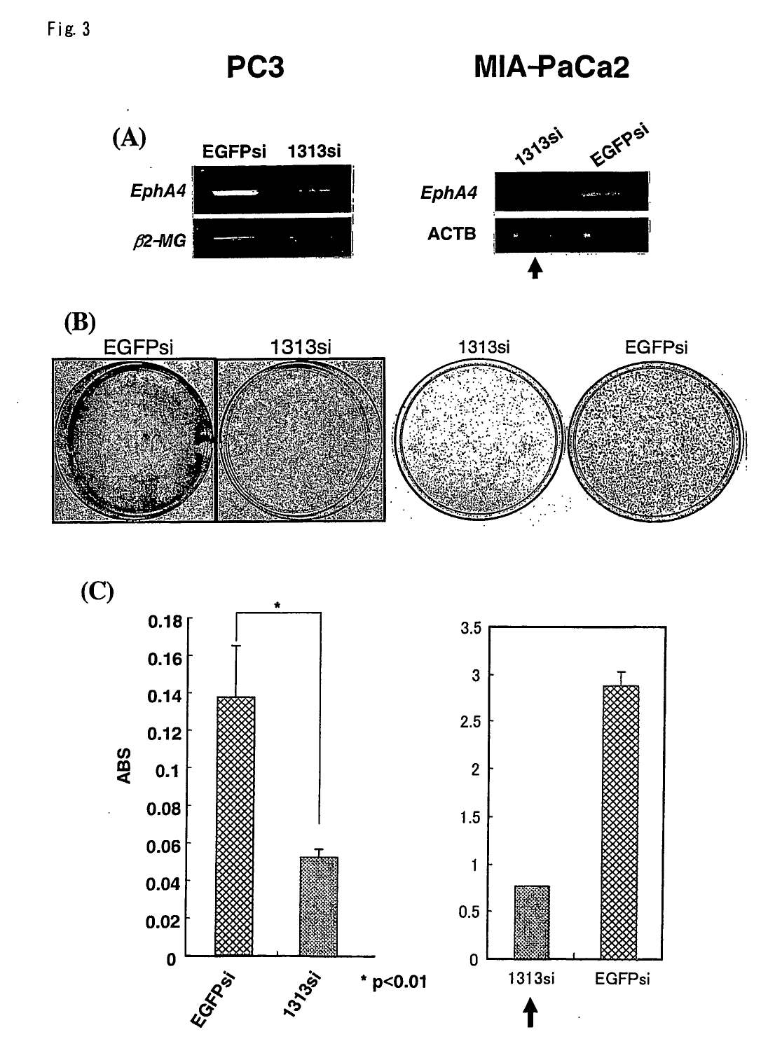

To investigate the growth or survival effect on prostate cancer of EphA4, we knocked down their endogenous expression specifically by mammalian vector-based RNA interference (RNAi) technique. The transfection of the siRNA-producing vectors resulted in reduction of the endogenous expression in some designed siRNA for EphA4 (FIG. 3A). The knocking-down effect by the siRNA on the transcript of EphA4 resulted in drastic growth suppression in colony formation assay and MTT assay (FIGS. 3B and 3C). These findings demonstrate that over-expression of EphA4 in prostate cancer cells is associated with cancer cell growth and they useful molecular targets of prostate cancer therapy.

In conclusion, we identified EphA4, a tyrosine kinase receptor over-expressed in prostate cancer cells, not in non-invasive precursor PINs, and it is associated with cancer cell growth, demonstrating that this tyrosine kinase receptor is an ideal molecular target of small molecules or antibodies for prostate cancer treatment.

### EXAMPLE 2

1. General Methods

Cell Lines and Tissue Specimens

Human Pancreatic cell lines PK45P, KLM1 and MIA-PaCa2 (ATCC Number: CRL-1420) were obtained from the Cell Resource Center for Biomedical Research, Institute of Development, Aging and Cancer, Tohoku University. All these cells are publicly available.

Isolation of Over-Expressing Genes in PDACa Cells by Using cDNA Microarray

Fabrication of the cDNA microarray slides has been described (Ono K, Tanaka T, Tsunoda T, Kitahara O, Kihara C, Okamoto A, Ochiai K, Takagi T, and Nakamura Y. Cancer Res., 60: 5007-5011, 2000). For each analysis of expression profiles it was prepared duplicate sets of cDNA microarray slides containing approximately 27000 DNA spots, to reduce experimental fluctuation. Briefly, total RNA was purified from PDACa cells and normal pancreatic duct epithelium microdissected from 18 pancreatic cancer tissues. T7-based RNA amplification was carried out to obtain adequate RNA for microarray experiments. Aliquots of amplified RNA from PDACa cells and normal duct epithelium were labeled by reverse transcription with Cy5-dCTP and Cy3-dCTP, respectively (Amersham Biosciences). Hybridization, washing, and detection were carried out as described previously (Ono K, Tanaka T, Tsunoda T, Kitahara O, Kihara C, Okamoto A, Ochiai K, Takagi T, and Nakamura Y. Cancer Res., 60: 5007-5011, 2000). Subsequently, among the up-regulated genes, it was focused four genes, EphA4 because its expression ratio was greater than 5.0 in more than 50% of informative cancers and their expression level in normal vital major organs was relatively low according to the our previous data of gene expression in 29 normal human tissues (Saito-Hisaminato A, Katagiri T, Kakiuchi S, Nakamura T, Tsunoda T, Nakamura Y. Genome-wide profiling of gene expression in 29 normal human tissues with a cDNA microarray. DNA Res., 9: 35-45, 2002).

Semiquantitative RT-PCR for EphA4

RNA from the microdissected PDACa cells and normal pancreatic ductal epithelial cells were subject to two-round amplification by T7-based in vitro transcription (Epicentre Technologies) and synthesized to single-strand cDNA. It was prepared appropriate dilutions of each single-stranded cDNA for subsequent PCR amplification by monitoring β-actin (ACTB) and β2-MG as a quantitative control. The primer sequences the present inventors used were

| | |
|---|---|
| 5′-GAAGGCGTGGTCACTAAATGTAA-3′ | (SEQ ID NO:3) |
| and | |
| 5′-TTTAATTTCAGAGGGCGAAGAC-3′ | (SEQ ID NO:4) |
| for EphA4, | |
| 5′-CATCCACGAAACTACCTTCAACT-3′ | (SEQ.ID.NO.5) |
| and | |
| 5′-TCTCCTTAGAGAGAAGTGGGGTG-3′ | (SEQ.ID.NO.6) |
| for ACTB, | |
| 5′-CACCCCCACTGAAAAAGAGA-3′ | (SEQ ID NO:7) |
| and | |
| 5′-TACCTGTGGAGCAAGGTGC-3′ | (SEQ ID NO:8) |
| for β2-MG. | |

All reactions involved initial denaturation at 94° C. for 2 min followed by 21 cycles (for ACTB and β2-MG) or 28-32 cycles (for EphA4) at 94° C. for 30 s, 58° C. for 30 s, and 72° C. for 1 min, on a GeneAmp PCR system 9700 (PE Applied Biosystems).

Immunohistochemistry

Formalin-fixed and paraffin-embedded PDACa sections were immunostained using a rabbit anti-EphA4 (EphA4) polyclonal antibody (Santa Cruz Biotechnology) for EphA4 expression. Deparaffinized tissue sections were placed in 10 mM citrate buffer, pH 6.0, and heated to 108° C. in an autoclave for 15 minutes for antigen retrieval. Sections were incubated with a 1:10 dilution or a 1:100 dilution of primary antibody for EphA4, respectively, in a humidity chamber for an hour at room temperature, and developed with peroxidase labeled-dextran polymer followed by diaminobenzidine (DAKO Envision Plus System; DAKO Corporation, Carpinteria, Calif.). Sections were counterstained with hematoxylin. For negative controls, primary antibody was omitted.

Northern Blot Analysis

Human multiple-tissue Northern blots (Clontech) were hybridized with a [α-³²P] dCTP-labeled PCR product amplified by the primers described above. Pre-hybridization, hybridization and washing were performed according to the supplier's recommendations. The blots were auto-radiographed with intensifying screens at −80° C. for 5 days.

Construction of psiU6BX Plasmid

The DNA flagment encoding siRNA was inserted into the GAP at nucleotide 85-490 as indicated (−) in the following plasmid sequence (SEQ ID No: 15).

GACGGATCGGGAGATCTCCCGATCCCCTATGGTGCACTCTCAGTACAATC
TGCTCTGGATCCACTAGTAACGGCCGCCAGTGTGCTGGAATTCGGCTTGG
GGATCAGCGTTTGAGTAAGAGCCCGCGTCTGAACCCTCCGCGCCGCCCCG
GCCCCAGTGGAAAGACGCGCAGGCAAAACGCACCACGTGACGGAGCGTGA
CCGCGCGCCGAGCGCGCGCCAAGGTCGGGCAGGAAGAGGGCCTATTTCCC
ATGATTCCTTCATATTTGCATATACGATACAAGGCTGTTAGAGAGATAAT
TAGAATTAATTTGACTGTAAACACAAAGATATTAGTACAAAATACGTGAC
GTAGAAAGTAATAATTTCTTGGGTAGTTTGCAGTTTTAAAATTATGTTTT
AAAATGGACTATCATATGCTTACCGTAACTTGAAAGTATTTCGATTTCTT
GGCTTTATATATCTTGTGGAAAGGACGAAACACC------TTTTTACATC
AGGTTGTTTTTCTGTTTGGTTTTTTTTTTACACCACGTTTATACGCCGGT
GCACGGTTTACCACTGAAAACACCTTTCATCTACAGGTGATATCTTTTAA
CACAAATAAAATGTAGTAGTCCTAGGAGACGGAATAGAAGGAGGTGGGGC
CTAAAGCCGAATTCTGCAGATATCCATCACACTGGCGGCCGCTCGAGTGA
GGCGGAAAGAACCAGCTGGGGCTCTAGGGGGTATCCCCACGCGCCCTGTA
GCGGCGCATTAAGCGCGGCGGGTGTGGTGGTTACGCGCAGCGTGACCGCT
ACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCC
CTTTAGGGTTCCGATTTAGTGCTTTACGGCACCTCGACCCCAAAAAACTT
GATTAGGGTGATGGTTCACGTAGTGGGCCATCGCCCTGATAGACGGTTTT
TCGCCCTTTGACGTTGGAGTCCACGTTCTTTAATAGTGGACTCTTGTTCC
AAACTGGAACAACACTCAACCCTATCTCGGTCTATTCTTTTGATTTATAA
GGGATTTTGCCGATTTCGGCCTATTGGTTAAAAAATGAGCTGATTTAACA
AAAATTTAACGCGAATTAATTCTGTGGAATGTGTGTCAGTTAGGGTGTGG
AAAGTCCCCAGGCTCCCCAGCAGGCAGAAGTATGCAAAGCATGCATCTCA
ATTAGTCAGCAACCAGGTGTGGAAAGTCCCCAGGCTCCCCAGCAGGCAGA
AGTATGCAAAGCATGCATCTCAATTAGTCAGCAACCATAGTCCCGCCCCT
AACTCCGCCCATCCCGCCCCTAACTCCGCCCAGTTCCGCCCATTCTCCGC
CCCATGGCTGACTAATTTTTTTTATTTATGCAGAGGCCGAGGCCGCCTCT
GCCTCTGAGCTATTCCAGAAGTAGTGAGGAGGCTTTTTTGGAGGCCTAGG
CTTTTGCAAAAAGCTCCCGGGAGCTTGTATATCCATTTTCGGATCTGATC
AAGAGACAGGATGAGGATCGTTTCGCATGATTGAACAAGATGGATTGCAC
GCAGGTTCTCCGGCCGCTTGGGTGGAGAGGCTATTCGGCTATGACTGGGC
ACAACAGACAATCGGCTGCTCTGATGCCGCCGTGTTCCGGCTGTCAGCGC
AGGGGCGCCCGGTTCTTTTTGTCAAGACCGACCTGTCCGGTGCCCTGAAT
GAACTGCAGGACGAGGCAGCGCGGCTATCGTGGCTGGCCACGACGGGCGT
TCCTTGCGCAGCTGTGCTCGACGTTGTCACTGAAGCGGGAAGGGACTGGC
TGCTATTGGGCGAAGTGCCGGGGCAGGATCTCCTGTCATCTCACCTTGCT
CCTGCCGAGAAAGTATCCATCATGGCTGATGCAATGCGGCGGCTGCATAC
GCTTGATCCGGCTACCTGCCCATTCGACCACCAAGCGAAACATCGCATCG
AGCGAGCACGTACTCGGATGGAAGCCGGTCTTGTCGATCAGGATGATCTG
GACGAAGAGCATCAGGGGCTCGCGCCAGCCGAACTGTTCGCCAGGCTCAA
GGCGCGCATGCCCGACGGCGAGGATCTCGTCGTGACCCATGGCGATGCCT
GCTTGCCGAATATCATGGTGGAAAATGGCCGCTTTTCTGGATTCATCGAC
TGTGGCCGGCTGGGTGTGGCGGACCGCTATCAGGACATAGCGTTGGCTAC
CCGTGATATTGCTGAAGAGCTTGGCGGCGAATGGGCTGACCGCTTCCTCG
TGCTTTACGGTATCGCCGCTCCCGATTCGCAGCGCATCGCCTTCTATCGC
CTTCTTGACGAGTTCTTCTGAGCGGGACTCTGGGGTTCGAAATGACCGAC
CAAGCGACGCCCAACCTGCCATCACGAGATTTCGATTCCACCGCCGCCTT
CTATGAAAGGTTGGGCTTCGGAATCGTTTTCCGGGACGCCGGCTGGATGA
TCCTCCAGCGCGGGGATCTCATGCTGGAGTTCTTCGCCCACCCCAACTTG
TTTATTGCAGCTTATAATGGTTACAAATAAAGCAATAGCATCACAAATTT
CACAAATAAAGCATTTTTTTCACTGCATTCTAGTTGTGGTTTGTCCAAAC
TCATCAATGTATCTTATCATGTCTGTATACCGTCGACCTCTAGCTAGAGC
TTGGCGTAATCATGGTCATAGCTGTTTCCTGTGTGAAATTGTTATCCGCT
CACAATTCCACACAACATACGAGCCGGAAGCATAAAGTGTAAAGCCTGGG
GTGCCTAATGAGTGAGCTAACTCACATTAATTGCGTTGCGCTCACTGCCC
GCTTTCCAGTCGGGAAACCTGTCGTGCCAGCTGCATTAATGAATCGGCCA
ACGCGCGGGGAGAGGCGGTTTGCGTATTGGGCGCTCTTCCGCTTCCTCGC
TCACTGACTCGCTGCGCTCGGTCGTTCGGCTGCGGCGAGCGGTATCAGCT
CACTCAAAGGCGGTAATACGGTTATCCACAGAATCAGGGGATAACGCAGG
AAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCAC
AAAAATCGACGCTCAAGTCAGAGGTGGCGAAACCCGACAGGACTATAAAG
ATACCAGGCGTTTCCCCCTGGAAGCTCCCTCGTGCGCTCTCCTGTTCCGA
CCCTGCCGCTTACCGGATACCTGTCCCCCTTTCTCCCTTCGGGAAGCGTG
GCGCTTTCTCATAGCTCACGCTGTAGGTATCTCAGTTCGGTGTAGGTCGT
TCGCTCCAAGCTGGGCTGTGTGCACGAACCCCCCGTTCAGCCCGACCGCT
GCGCCTTATCCGGTAACTATCGTCTTGAGTCCAACCCGGTAAGACACGAC
TTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACA
CTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAG
CGGTTTTTTTGTTTGCAAGCAGCAGATTACGCGCAGAAAAAAAGGATCTC
AAGAAGATCCTTTGATCTTTTCTACGGGGTCTGACGCTCAGTGGAACGAA
AACTCACGTTAAGGGATTTTGGTCATGAGATTATCAAAAAGGATCTTCAC
CTAGATCCTTTTAAATTAAAAATGAAGTTTTAAATCAATCTAAAGTATAT
ATGAGTAAACTTGGTCTGACAGTTACCAATGCTTAATCAGTGAGGCACCT
ATCTCAGCGATCTGTCTATTTCGTTCATCCATAGTTGCCTGACTCCCCGT
CGTGTAGATAACTACGATACGGGAGGGCTTACCATCTGGCCCCAGTGCTG
CAATGATACCGCGAGACCCACGCTCACCGGCTCCAGATTTATCAGCAATA
AACCAGCCAGCCGGAAGGGCCGAGCGCAGAAGTGGTCCTGCAACTTTATC
CGCCTCCATCCAGTCTATTAATTGTTGCCGGGAAGCTAGAGTAAGTAGTT
CGCCAGTTAATAGTTTGCGCAACGTTGTTGCCATTGCTACAGGCATCGTG
GTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCT
CCTTCGGTCCTCCGATCGTTGTCAGAAGTAAGTTGGCCGCAGTGTTATCA
CTCATGGTTATGGCAGCACTGCATAATTCTCTTACTGTCATGCCATCCGT
AAGATGCTTTTCTGTGACTGGTGAGTACTCAACCAAGTCATTCTGAGAAT
AGTGTATGCGGCGACCGAGTTGCTCTTGCCCGGCGTCAATACGGGATAAT
ACCGCGCCACATAGCAGAACTTTAAAAGTGCTCATCATTGGAAAACGTTC
TTCGGGGCGAAAACTCTCAAGGATCTTACCGCTGTTGAGATCCAGTTCGA
TGTAACCCACTCGTGCACCCAACTGATCTTCAGCATCTTTTACTTTCACC
AGCGTTTCTGGGTGAGCAAAAACAGGAAGGCAAAATGCCGCAAAAAAGGG
AATAAGGGCGACACGGAAATGTTGAATACTCATACTCTTCCTTTTTCAAT
ATTATTGAAGCATTTATCAGGGTTATTGTCTCATGAGCGGATACATATTT
GAATGTATTTAGAAAAATAAACAAATAGGGGTTCCGCGCACATTTCCCCG
AAAAGTGCCACCTGACGTC

snRNA U6 gene is reported to be transcribed by RNA polymerase III, which produce short transcripts with uridines at the 3′ end. The genomic fragment of the snRNA U6 gene containing the promoter region was amplified by PCR using a set of primers,

5′-GGGGATCAGCGTTTGAGTAA-3′ (SEQ ID No: 16), and

5′-TAGGCCCCACCTCCTTCTAT-3′ (SEQ ID No: 17) and human placental DNA as a template. The product was purified and cloned into pCR plasmid vector using a TA cloning kit according to the supplier's protocol (Invitrogen). The BamHI, XhoI fragment containing the snRNA U6 gene was purified and cloned into nucleotide 1257 to 56 fragment of pcDNA3.1(+) plasmid, which was amplified by PCR with a set of primer, 5′-TGCGGATCCAGAGCAGATTGTACTGAGAGT-3′ (SEQ ID No: 18) and

5′-CTCTATCTCGAGTGAGGCGGAAAGAACCA-3′ (SEQ ID No: 19). The ligated DNA was used for a template of PCR with primers,

5′-TTTAAGCTTGAAGACTATTTTTACATCAGGTTGTTTTTCT-3′ (SEQ ID No: 20) and

5′-TTTAAGCTTGAAGACACGGTGTTTCGTCCTTTCCACA-3′ (SEQ ID No: 21). The product was digested with HindIII, which was subsequently self-ligated to produce psiU6BX vector plasmid. For the control, psiU6BX-EGFP was prepared by cloning double-stranded oligonucleotides of

5′-CACCGAAGCAGCACGACTTCTTCTTCAAGAGAGAAGAAGTCGTGC TGCTTC-3′ (SEQ ID No: 22) and

5′-AAAAGAAGCAGCACGACTTCTTCTCTCTTGAAGAAGAAGTCGTGCT GCTTC-3′ (SEQ ID No: 23) into the BbsI site in the psiU6BX vector.

siRNA-Expressing Constructs

The nucleotide sequence of the siRNAs were designed using an siRNA design computer program available from the Ambion website. (http://www.ambion.com/techlib/misc/siRNA_finder.html). Briefly, nucleotide sequences for siRNA synthesis are selected using the following protocol.

Selection of siRNA Target Sites:

1. Starting with the AUG start codon of the each gene transcript, scan downstream for an AA dinucleotide sequences. The occurrence of each AA and the 3′ adjacent 19 nucleotides are recorded as potential siRNA target sites. Tuschl et al. don't recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with binding of the siRNA endonuclease complex.

2. The potential target sites are compared to the appropriate genome database (human, mouse, rat, etc.) to eliminate target sequences with significant homology to other coding sequences.

3. Qualifying target sequences are selected for synthesis. Several target sequences along the length of the gene are selected for evaluation.

The oligonucleotides used for siRNAs of EphA4 are shown below. Each oligonucleotide is a combination of a sense nucleotide sequence and an antisense nucleotide sequence of the target sequence. The nucleotide sequences of the hairpin loop structure and target sequence are shown in SEQ ID NO:14 and SEQ ID NO:10, respectively (endonuclease recognition cites are eliminated from each hairpin loop structure sequence).

Insert Sequence of siRNA for EphA4

1313si:

(SEQ ID NO:12)

5′-CACCGCAGCACCATCATCCATTGTTCAAGAGACAATGGATGATGGTGC
TGC-3′
and

(SEQ ID NO:13)

5′-AAAAGCAGCACCATCATCCATTGTCTCTTGAACAATGGATGATGGTG
CTGC-3′

Insert Sequence of siRNA for Control

EGFPsi: (control)

(SEQ ID NO:22)

5′-CACCGAAGCAGCACGACTTCTTCTTCAAGAGAGAAGAAGTCGTGCTG
CTTC-3′
and

(SEQ ID NO:23)

5′-AAAAGAAGCAGCACGACTTCTTCTCTCTTGAAGAAGAAGTCGTGCTG
CTTC-3′

Sequence ID NO of each sequences are listed in Table 1

| | | | | | hairpin | target SEQ | |
|---|---|---|---|---|---|---|---|
| gene | siRNA | effect | insert seq SEQ ID NO | | siRNA | ID NO | position |
| EphA4 | 1313si | + | 12 | 13 | 14 | 10 | 1357-1375 |
| control | EGFPsi | − | 22 | 23 | | 11 | |

Colony Formation/MTT Assay

Human PDACa cell lines among PK45P, KLM1 and MIA-PaCa2, were plated onto 10-cm dishes (5×10⁵ cells/dish) and transfected with psiU6BX containing EGFP target sequence (EGFP) and psiU6BX containing target sequence using Lipofectamine 2000 (Invitrogen) or FuGENE6 (Roche), according to manufacture's instruction. Cells were selected by 500 mg/ml Geneticin for one week, and preliminary cells were harvested 8 hours after transfection and analyzed by RT-PCR to validate knockdown effect on EphA4. The primers of RT-PCR were the same ones described above. These cells were also stained by Giemsa solution and performed MTT assay to evaluate the colony formation and the cell number, respectively.

2. Reduction of the Expression of the Genes EphA4 and Growth Suppression of Cancer Cells by siRNA

In previous study, it was generated precise expression profiles of PDACa by combining laser microdissection with genome-wide cDNA microarrays with 27,000 genes spotted. The present inventors identified more than 200 genes as up-regulated genes in PDACa cells comparing with the expression pattern of normal pancreatic ductal epithelium that was thought to be the origin of PDACa (Nakamura T, Furukawa Y, Nakagawa H, Tsunoda T, Ohigashi H, Murata K, Ishikawa O, Ohgaki, Kashimura N, Miyamoto M, Hirano S, Kondo S, Katoh H, Nakamura Y, and Katagiri T. Genome-wide cDNA microarray analysis of gene-expression profiles in pancreatic cancers using populations of tumor cells and normal ductal epithelium cells selected for purity by laser microdissection. Oncogene, 2004 Feb. 9, Epub ahead of print). Based on these expression profile of PDACa cells, the present inventors selected one over-expressing gene, EphA4 and validated this overexpression in PDACa by immunohistochemistry (FIG. 1B). Their products are supposed to be cell-surface membrane proteins that are ideal molecule target for drug design and antibody therapy against cancer. Clinical trials approved that Trastuzumab (Herceptin), a humanized monoclonal antibody against ERBB2 (Her2) is effective for subsets of metastatic breast cancer with HER2 over-expressed, and cell-surface molecules that mediates signaling process necessary for essential cellular functions and for maintaining the malignant phenotypes are now most promising targets for cancer therapy (Pegram M, and Slamon D J. Biological rationale for Her2/neu as a target for monoclonal antibody therapy. Semin. Oncology, 27 (suppl 9): 13-19, 2000). Drug design targeting these membrane molecules can be approached both by blocking their growth-promoting signals and/or by modulating ADCC activity in the same way with Trastuzumab.

EphA4 (Genbank Accession No. NM_{—}004438; SEQ ID No.1,2)

The present inventors validated EphA4 over-expression in PDACa by RT-PCR and immunohistochemistry (FIG. 1B), but in pancreatic cancer tissues, the ligand of EphA4 is unknown. Northern blot analysis (FIG. 2) showed that EphA4 was abundant in testis, not in central nervous system and other major organs. Recently the antibody targeting against other Eph receptor family member, EphA42 that is also over-expressed in several cancers, was reported to inhibit breast cancer cell growth in vitro and in vivo (Carles-Kinch K, Kilpatrick K E, Stewart J C, Kinch M S. Antibody targeting of the EphA42 tyrosine kinase inhibits malignant cell behavior. Cancer Res., 62:2840-2847, 2002). However, EphA42 is expressed ubiquitously in adult tissues, indicating much more possibility of toxicity in treatment of antibody therapy. To investigate the growth or survival effect of EphA4 on PDACa cells, the present inventors knocked down their endogenous expression of EphA4 specifically by siRNA in PDACa cell line. The transfection of the siRNA-producing vectors clearly resulted in reduction of the endogenous expression in one designed siRNA, 1313si, for EphA4 (FIG. 3A). This knocking-down effect by the siRNA on EphA4 mRNA resulted in drastic growth suppression in colony formation assay (FIG. 3B) and MTT assay (FIG. 3C). Considering its tyrosine kinase activity, membrane localization and its specific expression pattern, EphA4 is one the most ideal molecular targets for pancreatic cancer.

In conclusion, the present inventors identified four membrane-type molecules over-expressed in PDACa cells and all of them are likely to be associated with cancer cell growth, suggested these membrane-type molecules are ideal molecular targets for deadly pancreatic cancer treatment and antibodies against these membrane molecules are a useful therapeutic approach.

### INDUSTRIAL APPLICABILITY

The methods described herein are useful in the identification of additional molecular targets for prevention, and treatment of PRC and PADCa. The data reported herein add to a comprehensive understanding of PRC, facilitate development of novel diagnostic strategies, and provide molecular targets for therapeutic drugs and preventative agents. Such information contributes to a more profound understanding of prostatic tumorigenesis, and provides indicators for developing novel strategies for diagnosis, treatment, and ultimately prevention of PRC.

The present inventors also have shown that the cell growth is suppressed by small interfering RNA (siRNA) that specifically target of the EphA4 gene. Thus, siRNAs are useful for the development of anti-cancer pharmaceuticals. For example, agents that block the expression of EphA4 or prevent its activity find therapeutic utility as anti-cancer agents, particularly anti-cancer agents for the treatment of prostate cancer or pancreatic cancer, such as pancreatic ductal adenocarcinoma (PDACa).

All patents, patent applications, and publications cited herein are incorporated by reference in their entirety. Furthermore, while the invention has been described in detail and with reference to specific embodiments thereof, it will be apparent to one skilled in the art that various changes and modifications can be made therein without departing from the spirit and scope of the invention.

### REFERENCES

Abate-Shen, C., and Shen, M. M. Molecular genetics of prostate cancer. Genes & Dev., 14: 2410-2434, 2000.
Bostwick, D. G. Prostatic intraepithelial neoplasia. Curr. Urol. Rep., 1: 65-70, 2000.
DeMarzo, A. M., Nelson, W. G., Isaacs, W. B., and Epstein, J. I. Pathological and molecular aspects of prostate cancer. Lancet, 361: 955-964, 2003.
Gronberg, H. Prostate cancer epidemiology. Lancet, 361: 859-64, 2003.
Kullander, K., and Klein, R. Mechanisms and functions of Eph and ephrin signalling. Nat. Rev. Mol. Cell. Biol., 3:75-486, 2002.
McNeal, J. E., and Bostwick, D. G. Intraductal dysplasia: a premalignant lesion of the prostate. Hum. Pathol., 17: 64-71, 1986.
Montironi R, Mazzucchelli R, Scarpelli M. Precancerous lesions and conditions of the prostate: from morphological and biological characterization to chemoprevention. Ann. N.Y. Acad. Sci., 963: 169-184, 2002.
Nelson, G. N., and Wilding, G. Prostate cancer prevention agent: criteria and pipeline for candidate chemoprevention agents. Urology, 57, 56-63, 2001.
Ono, K., Tanaka, T., Tsunoda, T., Kitahara, O., Kihara, C., Okamoto, A., Ochiai, K., Takagi, T., and Nakamura, Y. Alterations of gene expression during colorectal carcinogenesis revealed by cDNA microarrays after laser-capture microdissection of tumor tissues and normal epithelia. Cancer Res., 60: 5007-5011, 2000.
Steiner, M. S. High grade prostatic intraepithelial neoplasia is a disease. Curr. Urol. Rep., 2: 195-198, 2001.
van der Kwast, T. H., Lopes, C., Santonja, C., Pihl, C. G., Neetens, I., Martikainen, P., Di Lollo, S., Bubendorf, L., Hoedemaeker, R. F.; Members of the pathology committee of the European Randomised Study of Screening for Prostate Cancer. Guidelines for processing and reporting of prostatic needle biopsies. J. Clin. Pathol., 56:336-40, 2003.
Emmert-Buck, M. R., Bonner, R. F., Smith, P. D., Chuaqui, R. F., Zhuang, Z., Goldstein, S. R., Weiss, R. A., and Liotta, L. A. Laser capture microdissection. Science, 274: 998-1001., 1996.
Carles-Kinch K, Kilpatrick K E, Stewart J C, Kinch M S. Antibody targeting of the EphA42 tyrosine kinase inhibits malignant cell behavior. Cancer Res., 62: 2840-2847, 2002.
Coffman K T, Hu M, Carles-Kinch K, Tice D, Donacki N, Munyon K, Kifle G, Woods R, Langermann S, Kiener P A, Kinch M S. Differential EphA42 epitope display on normal versus malignant cells. Cancer Res. 63:7907-7912, 2003.
Dancey J and Sausville E A. Issue and progress with protein kinase inhibitors for cancer treatment. Nat. Rev. Drug Discovery 2: 296-313, 2003.
Dodelet V C, Pasquale E B. Eph receptors and ephrin lignads: embryogenesis to tumorigenesis. Oncogene, 19: 5614-5619, 2000.
Gale N W and Yancopoulos G D. Growth factors acting via endothelial cell-specific receptor tyrosine kinases: VEGFs, angiopoietins, and ephrins in vascular development. Gene Dev., 13: 1055-1066, 1999.
Han M, Partin A W, Piantadosi S, Epstein J I, Walsh P C. J Urol, 166:16-419, 2001.
Kurai K K, Pasquale E B. ‘Eph7ective signaling: forward, reverse and crosstalk. J. Cell Sci., 116: 2823-2832, 2003.
Morgan B, Thomas A L, Drevs J, Hennig J, Buchert M, Jivan A, Horsfield M A, Mross K, Ball H A, Lee L, Mietlowski W, Fuxuis S, Unger C, O'Byrne K, Henry A, Cherryman G R, Laurent D, Dugan M, Marme D, Steward W P. Dynamic contrast-enhanced magnetic resonance imaging as a biomarker for the pharmacological response of PTK787/ZK 222584, an inhibitor of the vascular endothelial growth factor receptor tyrosine kinases, in patients with advanced colorectal cancer and liver metastases: results from two phase I studies. J. Clin. Oncol., 21:3955-3964, 2003.
Sharp P A. RNAi and double-strand RNA. Genes Dev. 1999 Jan. 15; 13(2):139-41.
Hammond S M, Bernstein E, Beach D, Hannon G J. An RNA-directed nuclease mediates post-transcriptional gene silencing in Drosophila cells. Nature. 2000 Mar. 16; 404(6775):293-6.
Hannon G J. RNA interference. Nature. 2002 Jul. 11; 418(6894):244-51.
Elbashir S M, Harborth J, Lendeckel W, Yalcin A, Weber K, Tuschl T. Duplexes of 21-nucleotide RNAs mediate RNA interference in cultured mammalian cells. Nature. 2001 May 24; 411(6836):494-8.
Miyagishi M, Taira K. U6 promoter-driven siRNAs with four uridine 3′ overhangs efficiently suppress targeted gene expression in mammalian cells. Nat. Biotechnol. 2002 May; 20(5):497-500
Brummelkamp T R, Bernards R, Agami R. A System for Stable Expression of Short Interfering RNAs in Mammalian Cells Science. 296(5567):550-553, Apr. 19, 2002.

1. A method of diagnosing PRC or a predisposition to developing PRC in a subject, comprising determining a level of expression of EphA4 in a patient derived biological sample, wherein an increase of said level compared to a normal control level of said gene indicates that said subject suffers from or is at risk of developing PRC.

2. The method of claim 1, wherein said increase is at least 10% greater than said normal control level.

3. The method of claim 1, wherein the expression level is determined by any one method select from group consisting of:
(a) detecting the mRNA of EphA4,
(b) detecting the protein encoded by EphA4, and
(c) detecting the biological activity of the protein encoded by EphA4.

4. The method of claim 1, wherein said level of expression is determined by detecting hybridization of EphA4 probe to a gene transcript of said patient-derived biological sample.

5. The method of claim 4, wherein said hybridization step is carried out on a DNA array.

6. The method of claim 1, wherein said biological sample comprises an epithelial cell.

7. The method of claim 1, wherein said biological sample comprises PRC cell.

8. The method of claim 7, wherein said biological sample comprises an epithelial cell from a PRC.

9. A method of screening for a compound for treating or preventing PRC, said method comprising the steps of:
a) contacting a test compound with a polypeptide encoded by EphA4;
b) detecting the binding activity between the polypeptide and the test compound; and
c) selecting a compound that binds to the polypeptide.

10. A method of screening for a compound for treating or preventing PRC, said method comprising the steps of:
a) contacting a candidate compound with a cell expressing EphA4; and
b) selecting a compound that reduces the expression level of EphA4.

11. The method of claim 10, wherein said cell comprises a prostate cancer cell.

12. A method of screening for a compound for treating or preventing PRC, said method comprising the steps of:
a) contacting a test compound with a polypeptide encoded by EphA4;
b) detecting the biological activity of the polypeptide of step (a); and
c) selecting a compound that suppresses the biological activity of the polypeptide in comparison with the biological activity detected in the absence of the test compound.

13. The method of claim 12, wherein the biological activity is tyrosine kinase activity.

14. A method of screening for compound for treating or preventing PRC, said method comprising the steps of:
a) contacting a test compound with a cell into which a vector comprising the transcriptional regulatory region of EphA4 genes and a reporter gene that is expressed under the control of the transcriptional regulatory region has been introduced,
b) measuring the expression or activity of said reporter gene; and
c) selecting a compound that reduces the expression or activity level of said reporter gene, as compared to a level in the absence of the test compound.

15. A method of treating or preventing PRC in a subject comprising administering to said subject an antisense composition, said composition comprising a nucleotide sequence complementary to a coding sequence of EphA4.

16. A method of treating or preventing PRC in a subject comprising administering to said subject a siRNA composition, wherein said composition reduces the expression of EphA4.

17. The method of claim 16, wherein said siRNA comprises a sense nucleic acid and an anti-sense nucleic acid of EphA4.

18. The method of claim 17, wherein the siRNA comprises a ribonucleotide sequence corresponding to a sequence consisting of SEQ ID NO: 10 as the target sequence.

19. The method of claim 18, said siRNA has the general formula 5′-[A]-[B]-[A′]-3′, wherein [A] is a ribonucleotide sequence corresponding to a sequence consisting of nucleotides of SEQ ID NO: 10.
[B] is a ribonucleotide sequence consisting of about 3 to about 23 nucleotides, and
[A′] is a ribonucleotide sequence consisting of the complementary sequence of [A].

20. The method of claim 16, wherein said composition comprises a transfection-enhancing agent.

21. A method of treating or preventing PRC in a subject comprising the step of administering to said subject a pharmaceutically effective amount of an antibody or fragment thereof that binds to a protein encoded by EphA4.

22. A method of treating or preventing PRC in a subject comprising administering to said subject a vaccine comprising a polypeptide encoded by EphA4 or an immunologically active fragment of said polypeptide, or a polynucleotide encoding the polypeptide.

23. A method of treating or preventing PRC in a subject, said method comprising the step of administering a compound that is obtained by the method according to any one of claims 9-14.

24. A composition for treating or preventing PRC, said composition comprising a pharmaceutically effective amount of an antisense polynucleotide or siRNA against a EphA4 as an active ingredient, and a pharmaceutically acceptable carrier.

25. The composition of claim 24, wherein said siRNA comprises the nucleotide sequence consisting of SEQ ID NO: 10 as the target sequence.

26. A composition for treating or preventing PRC, said composition comprising a pharmaceutically effective amount of an antibody or fragment thereof that binds to a protein encoded by EphA4 as an active ingredient, and a pharmaceutically acceptable carrier.

27. A composition for treating or preventing PRC, said composition comprising a pharmaceutically effective amount of the compound selected by the method of any one of claims 9-14 as an active ingredient, and a pharmaceutically acceptable carrier.

28. A method for treating or preventing pancreatic cancer in a subject comprising administering to said subject a composition comprising a siRNA of EphA4.

29. The method of claim 28, wherein said siRNA comprises a sense nucleic acid and an anti-sense nucleic acid of EphA4.

30. The method of claim 28, wherein the pancreatic cancer is an pancreatic ductal adenocarcinoma (PDACa).

31. The method of claim 29, wherein the siRNA comprises a ribonucleotide sequence corresponding to a sequence consisting of SEQ ID NO: 10 as the target sequence.

32. The method of claim 31, said siRNA has the general formula 5′-[A]-[B]-[A′]-3′, wherein [A] is a ribonucleotide sequence corresponding to a sequence consisting of nucleotides of SEQ ID NO: 10.
[B] is a ribonucleotide sequence consisting of about 3 to about 23 nucleotides, and
[A′] is a ribonucleotide sequence consisting of the complementary sequence consisting of [A].

33. The method of claim 28, wherein said composition comprises a transfection-enhancing agent.

34. A double-stranded molecule comprising a sense strand and an antisense strand, wherein the sense strand comprises a ribonucleotide sequence corresponding to a target sequence consisting of SEQ ID NO: 10, and wherein the antisense strand comprises a ribonucleotide sequence which is complementary to said sense strand, wherein said sense strand and said antisense strand hybridize to each other to form said double-stranded molecule, and wherein said double-stranded molecule, when introduced into a cell expressing the EphA4 gene, inhibits expression of said gene.

35. The double-stranded molecule of claim 34, wherein said target sequence comprises at least about 10 contiguous nucleotides from the nucleotide sequence consisting of SEQ ID NO: 1.

36. The double-stranded molecule of claim 35, wherein said target sequence comprises from about 19 to about 25 contiguous nucleotides from the nucleotide sequence consisting of SEQ ID NO: 1.

37. The double-stranded molecule of claim 36, wherein said double-stranded molecule is a single ribonucleotide transcript comprising the sense strand and the antisense strand linked via a single-stranded ribonucleotide sequence.

38. The double-stranded molecule of claim 35, wherein the double-stranded molecule is an oligonucleotide of less than about 100 nucleotides in length.

39. The double-stranded molecule of claim 38, wherein the double-stranded molecule is an oligonucleotide of less than about 75 nucleotides in length.

40. The double-stranded molecule of claim 39, wherein the double-stranded molecule is an oligonucleotide of less than about 50 nucleotides in length.

41. The double-stranded molecule of claim 40, wherein the double-stranded molecule is an oligonucleotide of less than about 25 nucleotides in length.

42. The double-stranded polynucleotide of claim 41, wherein the double stranded molecule is an oligonucleotide of between about 19 and about 25 nucleotides in length.

43. A vector encoding the double-stranded molecule of claim 35.

44. The vector of claim 43, wherein the vector encodes a transcript having a secondary structure and comprises the sense strand and the antisense strand.

45. The vector of claim 44, wherein the transcript further comprises a single-stranded ribonucleotide sequence linking said sense strand and said antisense strand.

46. A vector comprising a polynucleotide comprising a combination of a sense strand nucleic acid and an antisense strand nucleic acid, wherein said sense strand nucleic acid comprises nucleotide sequence consisting of SEQ ID NO: 10, and said antisense strand nucleic acid consists of a sequence complementary to the sense strand.

47. The vector of claim 46, wherein said polynucleotide has the general formula 5′-[A]-[B]-[A′]-3′
wherein [A] is a nucleotide sequence consisting of SEQ ID NO: 10; [B] is a nucleotide sequence consisting of about 3 to about 23 nucleotides; and [A′] is a nucleotide sequence complementary to [A].

48. A pharmaceutical composition for treating or preventing pancreatic cancer comprising a pharmaceutically effective amount of a small interfering RNA (siRNA) of EphA4 as an active ingredient, and a pharmaceutically acceptable carrier.

49. The pharmaceutical composition of claim 48, wherein the siRNA comprises a nucleotide sequence consisting of SEQ ID NO: 10 as the target sequence.

50. The composition of claim 49, wherein the siRNA has the general formula 5′-[A]-[B]-[A′]-3′
wherein [A] is a ribonucleotide sequence corresponding to a nucleotide sequence of SEQ ID NO: 10; [B] is a ribonucleotide sequence consisting of 3 to 23 nucleotides;
and [A′] is a ribonucleotide sequence complementary to [A].


SEQUENCE LISTING (23 sequences)

SEQ ID NO: 1 - DNA, 3468 bp, Homo sapiens
  CDS (43)..(3003)
ctgggatagaagcggcaggagcagcgttggcaccggcgaaccatggctgggattttctatttcgccctattttcgtgtctcttcgggatttgcgacgctgtcacaggttccagggtataccccgcgaatgaagttaccttattggattccagatctgttcagggagaacttgggtggatagcaagccctctggaaggagggtgggaggaagtgagtatcatggatgaaaaaaatacaccaatccgaacctaccaagtgtgcaatgtgatggaacccagccagaataactggctacgaactgattggatcacccgagaaggggctcagagggtgtatattgagattaaattcaccttgagggactgcaatagtcttccgggcgtcatggggacttgcaaggagacgtttaacctgtactactatgaatcagacaacgacaaagagcgtttcatcagagagaaccagtttgtcaaaattgacaccattgctgctgatgagagcttcacccaagtggacattggtgacagaatcatgaagctgaacaccgagatccgggatgtagggccattaagcaaaaaggggttttacctggcttttcaggatgtgggggcctgcatcgccctggtatcagtccgtgtgttctataaaaagtgtccactcacagtccgcaatctggcccagtttcctgacaccatcacaggggctgatacgtcttccctggtggaagttcgaggctcctgtgtcaacaactcagaagagaaagatgtgccaaaaatgtactgtggggcagatggtgaatggctggtacccattggcaactgcctatgcaacgctgggcatgaggagcggagcggagaatgccaagcttgcaaaattggatattacaaggctctctccacggatgccacctgtgccaagtgcccaccccacagctactctgtctgggaaggagccacctcgtgcacctgtgaccgaggctttttcagagctgacaacgatgctgcctctatgccctgcacccgtccaccatctgctcccctgaacttgatttcaaatgtcaacgagacatctgtgaacttggaatggagtagccctcagaatacaggtggccgccaggacatttcctataatgtggtatgcaagaaatgtggagctggtgaccccagcaagtgccgaccctgtggaagtggggtccactacaccccacagcagaatggcttgaagaccaccaaagtctccatcactgacctcctagctcataccaattacacctttgaaatctgggctgtgaatggagtgtccaaatataaccctaacccagaccaatcagtttctgtcactgtgaccaccaaccaagcagcaccatcatccattgctttggtccaggctaaagaagtcacaagatacagtgtggcactggcttggctggaaccagatcggcccaatggggtaatcctggaatatgaagtcaagtattatgagaaggatcagaatgagcgaagctatcgtatagttcggacagctgccaggaacacagatatcaaaggcctgaaccctctcacttcctatgttttccacgtgcgagccaggacagcagctggctatggagacttcagtgagcccttggaggttacaaccaacacagtgccttcccggatcattggagatggggctaactccacagtccttctggtctctgtctcgggcagtgtggtgctggtggtaattctcattgcagcttttgtcatcagccggagacggagtaaatacagtaaagccaaacaagaagcggatgaagagaaacatttgaatcaaggtgtaagaacatatgtggacccctttacgtacgaagatcccaaccaagcagtgcgagagtttgccaaagaaattgacgcatcctgcattaagattgaaaaagttataggagttggtgaatttggtgaggtatgcagtgggcgtctcaaagtgcctggcaagagagagatctgtgtggctatcaagactctgaaagctggttatacagacaaacagaggagagacttcctgagtgaggccagcatcatgggacagtttgaccatccgaacatcattcacttggaaggcgtggtcactaaatgtaaaccagtaatgatcataacagagtacatggagaatggctccttggatgcattcctcaggaaaaatgatggcagatttacagtcattcagctggtgggcatgcttcgtggcattgggtctgggatgaagtatttatctgatatgagctatgtgcatcgtgatctggccgcacggaacatcctggtgaacagcaacttggtctgcaaagtgtctgattttggcatgtcccgagtgcttgaggatgatccggaagcagcttacaccaccaggggtggcaagattcctatccggtggactgcgccagaagcaattgcctatcgtaaattcacatcagcaagtgatgtatggagctatggaatcgttatgtgggaagtgatgtcgtacggggagaggccctattgggatatgtccaatcaagatgtgattaaagccattgaggaaggctatcggttaccccctccaatggactgccccattgcgctccaccagctgatgctagactgctggcagaaggagaggagcgacaggcctaaatttgggcagattgtcaacatgttggacaaactcatccgcaaccccaacagcttgaagaggacagggacggagagctccagacctaacactgccttgttggatccaagctcccctgaattctctgctgtggtatcagtgggcgattggctccaggccattaaaatggaccggtataaggataacttcacagctgctggttataccacactagaggctgtggtgcacgtgaaccaggaggacctggcaagaattggtatcacagccatcacgcaccagaataagattttgagcagtgtccaggcaatgcgaacccaaatgcagcagatgcacggcagaatggttcccgtctgagccagtactgaataaactcaaaactcttgaaattagtttacctcatccatgcactttaattgaagaactgcactttttttacttcgtcttcgccctctgaaattaaagaaatgaaaaaaaaaaacaatatctgcagcgttgcttggtgcacagattgctgaaactgtggggcttacagaaatgactgccggtcatttgaatgagacctggaacaaatcgtttctcagaagtacttttctgttcatcaccagtctgtaaaatacatgtacctatagaaatagaacactgcctctgagttttgatgctgtatttgctgccagacactgagcttctgagacatccctgattctctctccatttggaattacaaccattgtattttgtttgtggcataaattacagtcatctgtctttcactggaatgaagaccatgcctaggaacattttttaaggactcagctgtgg

SEQ ID NO: 2 - protein, 986 aa, Homo sapiens
MAGIFYFALFSCLFGICDAVTGSRVYPANEVTLLDSRSVQGELGWIASPLEGGWEEVSIMDEKNTPIRTYQVCNVMEPSQNNWLRTDWITREGAQRVYIEIKFTLRDCNSLPGVMGTCKETFNLYYYESDNDKERFIRENQFVKIDTIAADESFTQVDIGDRIMKLNTEIRDVGPLSKKGFYLAFQDVGACIALVSVRVFYKKCPLTVRNLAQFPDTITGADTSSLVEVRGSCVNNSEEKDVPKMYCGADGEWLVPIGNCLCNAGHEERSGECQACKIGYYKALSTDATCAKCPPHSYSVWEGATSCTCDRGFFRADNDAASMPCTRPPSAPLNLISNVNETSVNLEWSSPQNTGGRQDISYNVVCKKCGAGDPSKCRPCGSGVHYTPQQNGLKTTKVSITDLLAHTNYTFEIWAVNGVSKYNPNPDQSVSVTVTTNQAAPSSIALVQAKEVTRYSVALAWLEPDRPNGVILEYEVKYYEKDQNERSYRIVRTAARNTDIKGLNPLTSYVFHVRARTAAGYGDFSEPLEVTTNTVPSRIIGDGANSTVLLVSVSGSVVLVVILIAAFVISRRRSKYSKAKQEADEEKHLNQGVRTYVDPFTYEDPNQAVREFAKEIDASCIKIEKVIGVGEFGEVCSGRLKVPGKREICVAIKTLKAGYTDKQRRDFLSEASIMGQFDHPNIIHLEGVVTKCKPVMIITEYMENGSLDAFLRKNDGRFTVIQLVGMLRGIGSGMKYLSDMSYVHRDLAARNILVNSNLVCKVSDFGMSRVLEDDPEAAYTTRGGKIPIRWTAPEAIAYRKFTSASDVWSYGIVMWEVMSYGERPYWDMSNQDVIKAIEEGYRLPPPMDCPIALHQLMLDCWQKERSDRPKFGQIVNMLDKLIRNPNSLKRTGTESSRPNTALLDPSSPEFSAVVSVGDWLQAIKMDRYKDNFTAAGYTTLEAVVHVNQEDLARIGITAITHQNKILSSVQAMRTQMQQMHGRMVPV

SEQ ID NO: 3 - DNA, 23 bp, Artificial
  An artificial synthesized primer sequence for RT-PCR
gaaggcgtggtcactaaatgtaa

SEQ ID NO: 4 - DNA, 22 bp, Artificial
  An artificial synthesized primer sequence for RT-PCR
tttaatttcagagggcgaagac

SEQ ID NO: 5 - DNA, 23 bp, Artificial
  An artificially synthesized primer sequence for RT-PCR
catccacgaaactaccttcaact

SEQ ID NO: 6 - DNA, 23 bp, Artificial
  An artificially synthesized primer sequence for RT-PCR
tctccttagagagaagtggggtg

SEQ ID NO: 7 - DNA, 20 bp, Artificial
  An artificial synthesized primer sequence for RT-PCR
cacccccactgaaaaagaga

SEQ ID NO: 8 - DNA, 19 bp, Artificial
  An artificial synthesized primer sequence for RT-PCR
tacctgtggagcaaggtgc

SEQ ID NO: 9 - DNA, 9 bp, Artificial
  An artificially synthesized spcer sequence for siRNA
ttcaagaga

SEQ ID NO: 10 - DNA, 19 bp, Artificial
  An artificial synthesized target sequence for siRNA
gcagcaccatcatccattg

SEQ ID NO: 11 - DNA, 19 bp, Artificial
  An artificial synthesized target sequence for siRNA
gaagcagcacgacttcttc

SEQ ID NO: 12 - DNA, 51 bp, Artificial
  An artificially synthesized sequence for siRNA
caccgcagcaccatcatccattgttcaagagacaatggatgatggtgctgc

SEQ ID NO: 13 - DNA, 51 bp, Artificial
  An artificially synthesized sequence for siRNA
aaaagcagcaccatcatccattgtctcttgaacaatggatgatggtgctgc

SEQ ID NO: 14 - DNA, 47 bp, Artificial
  siRNA hairpin design
gcagcaccatcatccattgttcaagagacaatggatgatggtgctgc

SEQ ID NO: 15 - DNA, 4863 bp, Artificial
  An artificially constructed plasmid sequence of siRNA  expression vector.
gacggatcgggagatctcccgatcccctatggtgcactctcagtacaatctgctctggatccactagtaacggccgccagtgtgctggaattcggcttggggatcagcgtttgagtaagagcccgcgtctgaaccctccgcgccgccccggccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacctttttacatcaggttgtttttctgtttggttttttttttacaccacgtttatacgccggtgcacggtttaccactgaaaacacctttcatctacaggtgatatcttttaacacaaataaaatgtagtagtcctaggagacggaatagaaggaggtggggcctaaagccgaattctgcagatatccatcacactggcggccgctcgagtgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 16 - DNA, 20 bp, artificial
  An artificially synthesized primer sequence
ggggatcagcgtttgagtaa

SEQ ID NO: 17 - DNA, 20 bp, artificial
  An artificially synthesized primer sequence
taggccccacctccttctat

SEQ ID NO: 18 - DNA, 30 bp, artificial
  An artificially synthesized primer sequence
tgcggatccagagcagattgtactgagagt

SEQ ID NO: 19 - DNA, 29 bp, artificial
  An artificially synthesized primer sequence
ctctatctcgagtgaggcggaaagaacca

SEQ ID NO: 20 - DNA, 40 bp, artificial
  An artificially synthesized primer sequence
tttaagcttgaagactatttttacatcaggttgtttttct

SEQ ID NO: 21 - DNA, 37 bp, artificial
  An artificially synthesized primer sequence
tttaagcttgaagacacggtgtttcgtcctttccaca

SEQ ID NO: 22 - DNA, 51 bp, artificial
  An artificially synthesized sequence for siRNA
caccgaagcagcacgacttcttcttcaagagagaagaagtcgtgctgcttc

SEQ ID NO: 23 - DNA, 51 bp, artificial
  An artificially synthesized sequence for siRNA
aaaagaagcagcacgacttcttctctcttgaagaagaagtcgtgctgcttc